(19) United States
(12) Patent Application Publication
(10) Pub. No.: US 2016/0158386 A1
(43) Pub. Date: Jun. 9, 2016

(54) MODEL OF COLORECTAL CANCER

(71) Applicant: GENENTECH, INC., South San Francisco, CA (US)

(72) Inventors: Erica JACKSON, South San Francisco, CA (US); Kevin LEONG, South San Francisco, CA (US)

(21) Appl. No.: 14/906,743

(22) PCT Filed: Jul. 23, 2014
(86) PCT No.: PCT/US14/47860

§ 371 Date: Jan. 21, 2016

Related U.S. Application Data
(60) Provisional application No. 61/857,638, filed on Jul. 23, 2013.
(60) Provisional application No. 61/954,788, filed on Mar. 18, 2014.

(51) Int. Cl. A61K 49/00; A01K 67/027

(52) U.S. Cl. CPC A61K 49/0008; A01K 67/0271; A01K 2207/12; A01K 2267/0331; A01K 2227/10

(57) ABSTRACT

A model of colorectal cancer that recapitulates the pathogenesis of the human disease is disclosed. Also provided are methods of generating the model of colorectal cancer, as well as methods of using the model to screen for compounds that inhibit tumorigenesis.

### FIELD OF THE INVENTION

The present invention relates generally to a clinically relevant model of colorectal cancer (CRC) and methods of using the model to screen for compounds that inhibit tumorigenesis.

### BACKGROUND OF THE INVENTION

Colorectal cancer (CRC) is the third most common cancer worldwide and the fourth most common cause of death. CRC accounts for over 9% of all cancer incidences. In 2013, it is estimated that 142,820 new CRC cases will be diagnosed in the United States and that 50,830 people will succumb to the disease (American Cancer Society: Cancer Facts and Figures 2013, Atlanta, Ga., 2013). The poor ratio of survival to incidence of CRC is due, at least in part, to the high percentage of cases that are diagnosed at an advanced stage. The overall five-year relative survival of patients with advanced stage metastatic CRC stands at 12.5% (Howlader et al. SEER Cancer Statistics Review, 1975-2010. NCI. Bethesda, Md., 2013).

Although xenograft, chemical-induced, and genetically-engineered models (e.g., mouse models) of CRC have been developed to study CRC, tumors in these models fail to reproducibly metastasize to the regional intestinal lymph nodes and liver, the target organs relevant to human CRC. Thus, there is an unmet need in the field for the development of a model of CRC that is capable of recapitulating the pathogenesis of the human disease. Such a model of CRC would be a valuable tool for testing therapeutics and developing novel treatment strategies.

### SUMMARY OF THE INVENTION

The present invention provides a model of colorectal cancer (CRC) that recapitulates the pathogenesis of the human disease, as well as methods for generating and using the model.

In a first aspect, the invention features a non-human mammal including a donor tumorigenic cell implant on the colonic mucosal surface, wherein implantation does not result in breach (e.g., opening, tear, rupture, or puncture) of the colon wall (i.e., the integrity of the deeper colon wall layers is maintained). In one embodiment, the donor tumorigenic cell implant is capable of invasive growth through the colon wall to the colonic serosal surface (e.g., growth resulting in penetration through the collagen IV-rich basement membrane of the muscularis externa to the serosal surface). In another embodiment, the invasive growth of the donor tumorigenic cell implant is characterized by metastases in common target organs (i.e., target metastatic organs or metastatic tissues) of CRC (e.g., human CRC), such as the intestinal lymph nodes, liver, or lungs. In another embodiment, the non-human mammal does not exhibit detectable tumor formation in the peritoneal cavity (e.g., peritoneal carcinomatosis) post-implantation. In another embodiment, the donor tumorigenic cell implant includes cells of a cancer cell line. The cancer cell line, in one embodiment, is a CRC cell line (e.g., HCT116). In another embodiment, the cancer cell line is a non-CRC cell line (e.g., a lung cancer cell line, a liver cancer cell line, a brain cancer cell line, a lymph node cancer cell line, a kidney cancer cell line, a stomach cancer cell line, a ovarian cancer cell line, a skin cancer cell line, a pancreatic cancer cell line, a thyroid cancer cell line, a prostate cancer cell line, or a breast cancer cell line, e.g., MDA-231). In another embodiment, the donor tumorigenic cell implant is an intact tumor, or fragment thereof. The intact tumor, or fragment thereof, in one embodiment, may be malignant (e.g., metastatic, regionally invasive, and/or distantly invasive). In another embodiment, the intact tumor, or fragment thereof, may be benign (e.g., non-metastatic and/or locally invasive). In some embodiments, the intact tumor, or fragment thereof, is an intact CRC tumor, or fragment thereof. In other embodiments, the intact tumor, or fragment thereof, is an intact non-CRC tumor, or fragment thereof (e.g., a breast cancer tumor, a lung cancer tumor, a liver cancer tumor, a brain cancer tumor, a lymph node cancer tumor, a kidney cancer tumor, a stomach cancer tumor, a ovarian cancer tumor, a skin cancer tumor, a pancreatic cancer tumor, a thyroid cancer tumor, or a prostate cancer tumor, or fragment thereof). In another embodiment, a subset (e.g., 5%, 10%, 11%, 12%, 13%, 14%, 15%, 16%, 17%, 18%, 19%, 20%, 21%, 22%, 23%, 24%, 25%, 26%, 27%, 28%, 29%, 30%, 35%, 40%, 45%, 50%, 55%, 60%, 65%, 70%, 75%, 80%, 85%, 90%, 95%, 96%, 97%, 98%, or 99% or more) of cells of the donor tumorigenic cell implant is capable of invasive growth. In other embodiments, growth of every (i.e., 100%) cell of the donor tumorigenic cell implant may be characterized as invasive growth. In another embodiment, the non-human mammal is a rodent, such as a mouse or a rat. In some embodiments, the rodent (e.g., mouse or rat) may be immunodeficient or immunocompromised. An immunodeficient mouse, in certain embodiments, may be a NOD/SCID mouse or a NOD/SCID interleukin-2 receptor gamma chain null (NSG) mouse. In other embodiments, the non-human mammal is wild-type and/or immune-competent (e.g., a wild-type or immune-competent rodent, e.g., a wild-type or immune-competent mouse or rat).

In a second aspect, the invention features a method for generating a non-human mammal (e.g., rodent, e.g., mouse or rat) of the first aspect (i.e., a non-human mammal (e.g., rodent) model for CRC), the method including exteriorizing the colonic mucosal surface of a host non-human mammal, implanting one or more tumorigenic cells onto the colonic mucosal surface, and re-inserting the exteriorized colon comprising the one or more implanted tumorigenic cells into the host non-human mammal.

In a third aspect, the invention features a method of screening for a compound that inhibits growth of tumorigenic cells (e.g., inhibits growth of tumorigenic cells by at least 20%, 25%, 30%, 35%, 40%, 45%, 50%, 55%, 60%, 65%, 70%, 75%, 80%, 85%, 90%, 95%, 96%, 97%, 98%, or 99% or greater, e.g., compared to an untreated or control-treated group), the method including contacting the donor tumorigenic cell implant of a non-human mammal of the invention with a candidate compound and determining whether the candidate compound inhibits growth of the tumorigenic cells, thereby identifying the candidate compound as a compound that inhibits growth of tumorigenic cells.

In a fourth aspect, the invention features a method of screening for an adjuvant that inhibits growth of tumorigenic cells (e.g., inhibits growth of tumorigenic cells by at least 20%, 25%, 30%, 35%, 40%, 45%, 50%, 55%, 60%, 65%, 70%, 75%, 80%, 85%, 90%, 95%, 96%, 97%, 98%, or 99% or greater, e.g., compared to an untreated or control-treated group), the method including: removing the donor tumorigenic cell implant from the colonic mucosal surface of a non-human mammal of the first aspect, administering to the non-human mammal a candidate compound, and determining whether the candidate compound inhibits growth of tumorigenic cells, thereby identifying the candidate compound as an adjuvant that inhibits growth of tumorigenic cells.

In one embodiment of the third or fourth aspect of the invention, the step of determining whether the candidate compound inhibits growth of tumorigenic cells includes evaluating the ability of the candidate compound to evoke at least one response (e.g., 1, 2, 3, 4, or 5 responses) selected from the group consisting of: reduction or stabilization in the number of tumorigenic cells; reduction or stabilization of tumor size; reduction or stabilization of tumor load; reduction or stabilization of tumorigenic cell invasiveness; and reduction or stabilization of tumor metastasis. In another embodiment of the third or fourth aspect, the candidate compound may be a small molecule, a peptide, a polypeptide, an antibody, an antibody fragment, or an immunoconjugate. In another embodiment of the third or fourth aspect, the donor tumorigenic cell implant may be capable of invasive growth through the colon wall to the colonic serosal surface (e.g., growth resulting in penetration through the collagen IV-rich basement membrane of the muscularis externa to the serosal surface). In other embodiments of the third or fourth aspect, invasive growth of the tumorigenic cells may be characterized by metastases in one or more (e.g., 1, 2, or 3 or more) common target organs (i.e., target metastatic organs or metastatic tissues) of CRC (e.g., human CRC), such as the intestinal lymph nodes, liver, or lungs. In other embodiments of the third or fourth aspect, the non-human mammal does not exhibit detectable tumor formation in the peritoneal cavity (e.g., peritoneal carcinomatosis) post-implantation. In another embodiment of the third or fourth aspect, the non-human mammal is a rodent, such as a mouse or rat.

### BRIEF DESCRIPTION OF THE DRAWINGS

The application file contains at least one drawing executed in color. Copies of this patent or patent application with color drawings will be provided by the Office upon request and payment of the necessary fee.

FIG. 1A shows images of the colon (top and middle panels) and liver (bottom panel) from a donor Apc^{Min/+}; Villin-Cre control mouse at 9 weeks of age. Colons were opened longitudinally and mucosal (top) and serosal (middle) views were imaged, with the anus positioned to the left. Arrows indicate colon polyps. Boxed areas of the liver have been enlarged.

FIG. 1B depicts images of the colon (top and middle panels) and liver (bottom panel) from a donor Apc^{Min/+}; Kras^{LSLG12D/+}; Villin-Cre mouse at 9 weeks of age. Colons were opened longitudinally and mucosal (top) and serosal (middle) views were imaged, with the anus positioned to the left. Arrows indicate colon polyps. Boxed areas of the liver have been enlarged.

FIG. 1C is a graph showing endogenous colon tumor burden in Apc^{Min/+}; Villin-Cre (n=5) and Apc^{Min/+}; Kras^{LSLG12D/+}; Villin-Cre (n=4) mice at 6 weeks of age. Each point represents data from an individual mouse. Means±s.e.m. are also shown. *P<0.05.

FIG. 1D is a graph showing Kaplan-Meier survival curves for Apc^{Min/+}; Villin-Cre (n=49) and Apc^{Min/+}; Kras^{LSLG12D/+}; Villin-Cre (n=14) mice. HR=hazard ratio.

FIG. 1E is a set of gross colon images (top panel: mucosal view; bottom panel: serosal view) from a host wild-type C57BL/6 mouse that has received a lumen implant of a single intact Apc^{Min/+}; Kras^{LSLG12D/+}; Villin-Cre colon tumor fragment from a donor subcutaneous allograft, harvested at 9 wpi. The boxed areas of the colon have been enlarged to the right of the respective image. Scale bars represent 3 mm.

FIGS. 1F and 1G are images of colons (top and middle panels) and livers (bottom panels) from two host wild-type C57BL/6 mice that have received lumen implants of a single intact Apc^{Min/+}; Kras^{LSLG12D/+}; Villin-Cre donor tumor, harvested at 63 wpi (F; host mouse #359; benign) or at 79 wpi (G; host mouse #590; malignant). Colon mucosal and/or serosal views were imaged with the anus positioned to the left. Arrows indicate implanted donor tumors. Boxed areas of the liver have been enlarged. In FIG. 1G, the boxed area of the colon has been enlarged to highlight a lymph node metastasis.

FIG. 1H is a table showing the incidence of benign versus malignant progression following lumen implantation of single intact Apc^{Min/+}; Kras^{LSLG12D/+}; Villin-Cre donor tumors into wild-type C57BL/6 host mouse colons.

FIG. 2 is a schematic diagram with representative images of the lumen implantation technique. Pre-implantation: the mouse is anesthetized by isofluorane inhalation and placed in a supine position, and the extremities are secured to a gauze-covered platform with adhesive tape. Hemostat insertion: a blunt hemostat is inserted into the anus, and the mucosa is gently clasped. Rectal prolapse induction: the hemostat is retracted from the anus, thus exteriorizing the mucosa. Tumor implantation: a donor tumor of approximately 10 mm³ is sutured onto the mucosal surface of the exteriorized colon. Tumor sutured: the suture ends are cut, leaving a donor tumor securely attached to the mucosa. Rectal prolapse reversal: a blunt gavage needle is used to re-insert the exteriorized colon together with the sutured donor tumor, thus reversing the rectal prolapse.

FIG. 3A is a series of endoscopy images following lumen implantation of a single Ape^{Min/+}; Kras^{LSLG12D/+}; Villin-Cre colon polyp from donor mouse #4700-260 into the colon of wildtype C57BL/6 host mouse #344, showing that the lumen-implanted Apc^{Min/+}; Kras^{LSLG12D/+}; Villin-Cre colon polyps remain benign. Serial images were captured from the host at the indicated times.

FIG. 3B is a series of endoscopy images following lumen implantation of a single Apc^{Min/+}; Kras^{LSLG12D/+}; Villin-Cre colon polyp from donor mouse #4700-260 into the colon of wildtype C57BL/6 host mouse #346, showing that the lumen-implanted Apc^{Min/+}; Kras^{LSLG12D/+}; Villin-Cre colon polyps remain benign. Serial images were captured from the host at the indicated times.

FIG. 4A is a series of images showing the time course of gross colorectal tumor development following lumen implantation. Colons from NOD/SCID mice bearing HCT116-DsRed lumen tumors were harvested at weekly intervals from 0 to 7 weeks post-implantation (wpi), opened longitudinally and imaged. Top and bottom panels show mucosal and serosal views of the colon, respectively, with the anus positioned to the left of every panel.

FIG. 4B is a series of endoscopy images following HCT116-DsRed lumen implantation. Serial images were captured from the same host from 0 to 4 wpi. Dotted line at 2 wpi indicates the perimeter of the implanted tumor.

FIG. 4C is a graph showing primary tumor volume following lumen implantation. Number of mice per time point: 0 wpi (n=3), 1 wpi (n=3), 2 wpi (n=6), 3 wpi (n=6), 4 wpi (n=11), 5 wpi (n=15), 6 wpi (n=11), 7 wpi (n=18). Data are represented by the mean and s.e.m.

FIG. 4D is a histological image of a host colon implanted with an HCT116-DsRed donor tumor at 1 wpi, showing haematoxylin and eosin (H&E) staining.

FIG. 4E is a histological image of a host colon implanted with an HCT116-DsRed donor tumor at 1 wpi, showing collagen IV (Col IV; green), DsRed (red), and 4,6-diamidino-2-phenylindole (DAPI; blue) staining.

FIG. 4F is a histological image of a host colon implanted with an HCT116-DsRed donor tumor at 3 wpi, showing H&E staining.

FIGS. 4G-4I are enlarged histological images of the indicated locations in FIG. 4F, each showing Col IV (green), DsRed (red) and DAPI (blue) staining.

FIG. 5A is a histological image of a colon from a NOD/SCID mouse stained with haematoxylin and eosin (H&E) immediately following lumen implantation of an HCT116-DsRed tumor fragment.

FIGS. 5B and 5C are enlarged histological images of the indicated locations in FIG. 5A, each showing H&E staining.

FIG. 5D is a histological image of a colon from a NOD/SCID mouse stained with Col IV (green), DsRed (red) and DAPI (blue) mouse immediately following lumen implantation of an HCT116-DsRed tumor fragment.

FIGS. 5E and 5F are enlarged histological images of the indicated locations in FIG. 5D, each showing Col IV (green), DsRed (red), and DAPI (blue) staining.

FIG. 5G is a set of images and graphs showing that tumor cell dissemination was not detectable at Day 1 post-transplantation. On Day 0, HCT116-DsRed donor tumor fragments were implanted onto the mucosal surface of host mouse colons (n=2). On Day 1 post-transplant, endoscopy was performed and images of the transplanted tumors were captured (top). Mice were then sacrificed and the liver, lungs, intestinal vascular tract, and blood were harvested and assessed by flow cytometry for DsRed⁺ cells. Negative controls consisted of tissue samples harvested from a wild-type mouse. Positive controls consisted of tissue samples containing HCT116-DsRed⁺ tumor cells. An average of 5×10⁶ viable cells were assessed by flow cytometry. Gates were established such that no DsRed⁺ cells within the respective negative control samples were detectable. Numbers within the gates denote the percentage of DsRed⁺ cells.

FIG. 6 is a series of images of colons from NOD/SCID mice bearing HCT116-DsRed lumen tumors showing the time course of colorectal tumor development following lumen implantation. The colons were harvested at weekly intervals from 0 to 7 wpi, opened longitudinally, fixed and sectioned, and stained by H&E (left column) or for Col IV (green), DsRed (red), and DAPI (blue) (right column). In all panels, the anus is positioned to the left with the lumen of the colon towards the top.

FIG. 7A is a set of images of a gross colon of a NOD/SCID mouse bearing an HCT116-DsRed lumen tumor at 7 wpi, with arrows indicating regional lymph node metastases; the bottom left panel showing histological staining by haematoxylin and eosin (H&E); and the blue- and red-boxed areas shown enlarged in the bottom middle and right panels, respectively, and stained for Col IV (green), DsRed (red), and DAPI (blue).

FIG. 7B is a set of images of a liver of a NOD/SCID mouse bearing an HCT116-DsRed lumen tumor at 7 wpi, with arrows indicating metastases; the middle panel showing histological staining by H&E, the dotted line indicating the perimeter of a metastatic nodule; and the boxed area shown enlarged in the right panel and stained for Col IV (green), DsRed (red), and DAPI (blue).

FIG. 7C is a set of images of lungs of a NOD/SCID mouse bearing an HCT116-DsRed lumen tumor at 7 wpi, with arrows indicating metastases; the middle panel showing histological staining by H&E, arrows indicating metastatic nodules; and the boxed area shown enlarged in the right panel and stained for Col IV (green), DsRed (red), and DAPI (blue).

FIG. 7D is a graph showing the number of macroscopic metastases within the intestinal lymph nodes, liver, and lungs following lumen implantation of an HCT116-DsRed tumor in NOD/SCID mice. Number of mice per time point: 0 wpi (n=14), 1 wpi (n=3), 2 wpi (n=6), 3 wpi (n=6), 4 wpi (n=11), 5 wpi (n=15), 6 wpi (n=20), 7 wpi (n=18). Each point represents data from an individual mouse. Means±s.e.m. are also shown.

FIG. 7E is a graph showing the DsRed-positive tumor cell burden within the intestinal lymph nodes, liver, and lungs following lumen implantation of an HCT116-DsRed tumor in NOD/SCID mice. Data are expressed as the number of DsRed-positive tumor cells per I×10⁶ viable events. Number of mice per time point: 0 wpi (n=14), 1 wpi (n=3), 2 wpi (n=3), 3 wpi (n=3), 4 wpi (n=8), 5 wpi (n=8), 6 wpi (n=13),7 wpi (n=14). Each point represents data from an individual mouse. Means±s.e.m. are also shown.

FIG. 7F is a set of images of a liver of a NOD/SCID mouse bearing an HCT116-DsRed lumen tumor at 3 wpi, with the middle panel showing histological staining by H&E, the right panel showing staining for Col IV (green), DsRed (red), and DAPI (blue), and arrows indicating DsRed-positive disseminated tumor cells.

FIG. 7G is a set of images of lungs of a NOD/SCID mouse bearing an HCT116-DsRed lumen tumor at 3 wpi, with the middle panel showing histological staining by H&E, the right panel showing staining for Col IV (green), DsRed (red), and DAPI (blue), arrows indicating DsRed-positive disseminated tumor cells, and arrowheads indicating autofluorescent macrophages.

FIG. 8A is a histological image of a colon stained with H&E from a NOD/SCID mouse bearing a lumen-implanted HCT116-DsRed tumor at 6 wpi, showing that lumen-implanted colorectal tumors exhibit locoregional spread as well as hematogenous/lymphatic/perineural invasion, and generate macroscopic lymph node metastases.

FIG. 8B is an enlarged histological image of the indicated location in FIG. 8A, stained with H&E, demonstrating locoregional spread distal to the primary tumor indicated by arrows.

FIG. 8C is an enlarged histological image of the indicated location in FIG. 8A, stained with H&E, demonstrating locoregional spread of a tumor migratory front into the normal mucosa outlined by a dotted line.

FIG. 8D is an enlarged histological image of the indicated location in FIG. 8A, stained with H&E, demonstrating muscularis externa penetration.

FIG. 8E is an enlarged histological image of the indicated location in FIG. 8A, stained with H&E, demonstrating primary tumor viability.

FIG. 8F is an enlarged histological image of the indicated location in FIG. 8A, stained with H&E, demonstrating locoregional spread proximal to the primary tumor indicated by arrows.

FIG. 8G is an enlarged histological image of the indicated location in FIG. 8A, stained with H&E, demonstrating hematogenous invasion indicated by an arrow.

FIG. 8H is an enlarged histological image of the indicated location in FIG. 8A, stained with H&E, demonstrating perineural invasion indicated by arrows.

FIG. 8I is an enlarged histological image of the indicated location in FIG. 8A, stained with H&E, demonstrating regional lymph node colonization.

FIG. 8J is an enlarged histological image of the indicated location in FIG. 8A, stained with H&E, demonstrating lymphatic invasion indicated by an arrow.

FIG. 9A is a series of images showing the time course of gross colorectal tumor development following lumen implantation. Colons from NOD/SCID mice bearing LS174T-DsRed lumen tumors were harvested from 0 to 8 wpi, opened longitudinally and imaged. Top and bottom panels show mucosal and serosal views of the colon, respectively, with the anus positioned to the left of every panel. Arrows indicate intestinal lymph node metastases.

FIG. 9B is a graph showing primary tumor volume following lumen implantation of LS174T-DsRed tumors in NOD/SCID mice. Number of mice per time point: 0 wpi (n=9), 1 wpi (n=3), 2 wpi (n=3), 3 wpi (n=3), 4 wpi (n=3), 5 wpi (n=3), 6 wpi (n=3), 7 wpi (n=4), 8 (n=12). Data are represented by the mean and s.e.m.

FIG. 9C is an image of a liver from a mouse bearing a lumen-implanted LS174T-DsRed tumor at 7 wpi. Arrows indicate metastases.

FIG. 9D is an image of lungs from a mouse bearing a lumen-implanted LS174T-DsRed tumor at 8 wpi. Arrow indicates a metastatic outgrowth.

FIG. 9E is a graph showing the number of macroscopic metastases within the intestinal lymph nodes, liver and lungs following lumen implantation of LS174T-DsRed tumors in NOD/SCID mice. Number of mice per time point: 0 wpi (n=9), 1 wpi (n=3), 2 wpi (n=3), 3 wpi (n=3), 4 wpi (n=3), 5 wpi (n=3), 6 wpi (n=3), 7 wpi (n=4), 8 wpi (n=12). Each point represents data from an individual mouse. Means±s.e.m. are also shown.

FIG. 10A is an image of a colon from a mouse bearing a lumen-implanted human stage II patient colorectal tumor, showing that lumen-implanted stage II patient tumors remain non-metastatic and benign.

FIG. 10B is an image of a colon from a mouse bearing a lumen-implanted human stage III patient colorectal tumor, showing that lumen-implanted stage III patient tumors give rise to lymph node metastases.

FIG. 10C is a graph showing the number of macroscopic metastases that result from stage II and stage III donor tumors. Each point represents data from an individual mouse. Means±s.e.m. are also shown. ****P<0.0001.

FIG. 11A is a graph showing the number of macroscopic metastases within various organs following lumen or s.c. implantation of HCT116-DsRed tumors into NOD/SCID or NSG mice. Number of mice: NOD/SCID lumen (n=22 for liver, lungs, lymph nodes; n=4 for adrenal gland, kidney, spleen, brain); NOD/SCID s.c. (n=10); NSG lumen (n=16); NSG s.c. (n=10). Each point represents data from an individual mouse. Means±s.e.m. are also shown. *P<0.05; **p<0.01; **P<0.001; ****P<0.0001. n.s.=not significant. n.d.=not determined.

FIG. 11B is a graph showing the DsRed-positive tumor cell burden within various organs following lumen or s.c. implantation of HCT116-DsRed tumors into NOD/SCID or NSG mice. Data are expressed as the number of DsRed-positive tumor cells per 1×10⁶ viable events. Number of mice: NOD/SCID lumen (n=13 for liver, lungs, lymph nodes; n=4 for adrenal gland, kidney, spleen, brain, bone marrow); NOD/SCID s.c. (n=10); NSG lumen (n=7 for liver, lungs, lymph nodes; n=3 for adrenal gland, kidney, spleen, brain, bone marrow); NSG s.c. (n=10). Each point represents data from an individual mouse. Means±s.e.m. are also shown. *P<0.05; *P<0.01; ***P<0.001; ****P<0.0001. n.s.=not significant. n.d.=not determined.

FIG. 11C is an image of a gross colon of an NSG mouse bearing an HCT116-DsRed lumen tumor at 7 wpi, with arrows indicating the primary tumor and regional lymph node metastases.

FIG. 11D is a histological image of a colon of an NSG mouse bearing an HCT116-DsRed lumen tumor at 7 wpi, the colon stained with H&E, and boxed area shown enlarged in the right panel and stained for Col IV (green), DsRed (red), and DAPI (blue). Arrows indicate regional lymph node metastases.

FIG. 11E are images of various organs from an NSG mouse bearing an HCT116-DsRed lumen tumor at 7 wpi, with liver metastases clearly evident and lung metastases indicated by arrows.

FIG. 11F is a histological image of a liver of an NSG mouse bearing an HCT116-DsRed lumen tumor at 7 wpi, the liver stained with H&E (left panel) and the for Col IV (green), DsRed (red), and DAPI (blue) (right panel). Dotted lines indicate the perimeter of liver metastatic nodules.

FIG. 11G is a histological image of lungs from an NSG mouse bearing an HCT116-DsRed lumen tumor at 7 wpi, the lungs stained with H&E (left panel) and the for Col IV (green), DsRed (red) and DAPI (blue) (right panel).

FIG. 11H are images of the liver and lungs of an NSG mouse bearing an HCT116-DsRed s.c. tumor at 7 wpi.

FIG. 11I is an image of the associated primary s.c. tumor of the NSG mouse of FIG. 11H.

FIG. 11J is a graph showing the circulating tumor cell number at 6-7 wpi following lumen or s.c. implantation of HCT116-DsRed tumors into NOD/SCID or NSG mice. Data are represented by the mean and s.e.m., and are expressed as the number of DsRed-positive tumor cells in the blood per 1×10⁶ viable events. Number of mice: NOD/SCID lumen (n=12); NOD/SCID s.c. (n=9); NSG lumen (n=9); NSG s.c. (n=10). **P<0.01; ***P<0.001.

FIG. 12A are images of the indicated organs from a NOD/SCID mouse bearing a lumen-implanted HCT116-DsRed tumor at 7 wpi.

FIG. 12B are images of the liver and lungs from a NOD/SCID mouse bearing an s.c.-implanted HCT116-DsRed tumor at 7 wpi.

FIG. 12C is a graph showing the primary tumor volume following s.c. implantation of HCT116-DsRed tumor cells (n=10). Data are represented by the mean±s.e.m.

FIG. 13A is a histological image of a lumen-implanted HCT116-DsRed tumor at 6 wpi, stained with H&E.

FIG. 13B is a histological image of an s.c.-implanted HCT116-DsRed tumor at 6 wpi, stained with H&E.

FIG. 13C is a histological image of a lumen-implanted HCT116-DsRed tumor at 6 wpi, stained with MECA-32 (green) and DAPI (blue).

FIG. 13D is a histological image of an s.c.-implanted HCT116-DsRed tumor at 6 wpi, stained with MECA-32 (green) and DAPI (blue).

FIG. 13E is a graph showing vascular density of HCT116-DsRed tumors implanted into the lumen (n=7) or s.c. (n=7) at 6 wpi. Data are represented by the mean and s.e.m., and are expressed as a ratio of the MECA-32-positive vascular area over the total DAPI-positive viable tumor area×100. *P<0.05.

FIG. 14A are graphs showing correlations between lymph node metastatic burden and liver metastatic burden in NOD/SCID mice bearing lumen-implanted HCT116-DsRed tumors from 1-7 wpi. Data are the total number of macroscopic liver metastases versus either the total number of macroscopic lymph node metastases (top) or the total number of DsRed⁺tumor cells within the lymph nodes (bottom).

FIG. 14B are graphs showing correlations between lymph node metastatic burden and liver metastatic burden in NSG mice bearing lumen-implanted HCT116-DsRed tumors from 5-7 wpi. Data are the total number of macroscopic liver metastases versus either the total number of macroscopic lymph node metastases (top) or the total number of DsRed⁺tumor cells within the lymph nodes (bottom).

FIG. 14C are gross images of colons of NOD/SCID mice bearing lumen-implanted HCT116-DsRed tumors at 6-7 wpi following the indicated antibody treatments. Arrowheads indicate intestinal lymph node metastases.

FIG. 14D is a graph showing the primary tumor volumes of NOD/SCID mice bearing lumen-implanted HCT116-DsRed tumors at 6-7 wpi following the indicated antibody treatments or no antibody treatment. Number of mice per treatment condition: untreated (n=22), anti-VEGF-A (n=9), anti-VEGF-C (n=8), anti-VEGF-A/C (n=10). Each point represents data from an individual mouse. Means±s.e.m. are also shown. *P<0.05; **P<0.01; ***P<0.001: ****P<0.0001. n.s.=not significant.

FIG. 14E is a graph showing the number of lymph node macroscopic metastases of NOD/SCID mice bearing lumen-implanted HCT116-DsRed tumors at 6-7 wpi following the indicated antibody treatments or no antibody treatment. Number of mice per treatment condition: untreated (n=22), anti-VEGF-A (n=9), anti-VEGF-C (n=8), anti-VEGF-A/C (n=10). Each point represents data from an individual mouse. Means±s.e.m. are also shown. *P<0.05; **P<0.01; ***P<0.001; ****P<0.0001. n.s.=not significant.

FIG. 14F is a graph showing the number of liver macroscopic metastases of NOD/SCID mice bearing lumen-implanted HCT116-DsRed tumors at 6-7 wpi following the indicated antibody treatments or no antibody treatment. Number of mice per treatment condition: untreated (n=22), anti-VEGF-A (n=9), anti-VEGF-C (n=8), anti-VEGF-A/C (n=10). Each point represents data from an individual mouse. Means±s.e.m. are also shown. *P<0.05; **P<0.01; ***P<0.001; ****P<0.0001. n.s.=not significant.

FIG. 14G is a set of gross images of the livers of NOD/SCID mice bearing lumen-implanted HCT116-DsRed tumors at 6-7 wpi following the indicated antibody treatments. Arrowheads indicate liver metastases.

FIG. 14H is a graph showing DsRed-positive tumor cell burden within the liver alongside control analyses from non-tumor-bearing mice (n=8), with data expressed as the number of DsRed-positive tumor cells per 1×10⁶ viable events, for NOD/SCID mice bearing lumen-implanted HCT116-DsRed tumors at 6-7 wpi following the indicated antibody treatments or no antibody treatment. Number of mice per treatment condition: untreated (n=13), anti-VEGF-A (n=9), anti-VEGF-C (n=8), anti-VEGF-A/C (n=9). Each point represents data from an individual mouse. Means±s.e.m. are also shown. *P<0.05; **P<0.01; ***P<0.001; ****P<0.0001. n.s.=not significant.

FIG. 14I is a contingency analysis comparing the number of NOD/SCID mice bearing lumen-implanted HCT116-DsRed tumors with or without liver macrometastases at 6-7 wpi, with data expressed as the percentage of mice in each category. Number of mice per treatment condition: untreated (n=13), anti-VEGF-A (n=9), anti-VEGF-C (n=8), anti-VEGF-NC (n=9). Means±s.e.m. are also shown. *P<0.05; **P<0.01; ***P<0.001; ****P<0.0001. n.s.=not significant.

FIG. 14J is a contingency analysis comparing the number of NOD/SCID mice bearing lumen-implanted HCT116-DsRed tumors with or without liver micrometastatic DsRed⁺cells at 3 wpi, prior to macroscopic metastasis manifestation, with data expressed as the percentage of mice in each category. Means±s.e.m. are also shown. *P<0.05; **P<0.01; ***P<0.001; ****P<0.0001. n.s.=not significant.

FIG. 14K is a graph showing the number of lymph node macroscopic metastases of NOD/SCID mice bearing lumen-implanted LS174T-DsRed tumors at 8 wpi following the indicated antibody treatments or no antibody treatment. Number of mice per treatment condition: untreated (n=7), anti-VEGF-A (n=3), anti-VEGF-C (n=13), anti-VEGF-A/C (n=5). Each point represents data from an individual mouse. Means±s.e.m. are also shown. *P<0.05. n.s.=not significant.

FIG. 14L is a contingency analysis comparing the number of NOD/SCID mice bearing lumen-implanted LS174T-DsRed tumors with or without liver macrometastases at 8 wpi, with data expressed as the percentage of mice in each category. Number of mice per treatment condition: untreated (n=7), anti-VEGF-A (n=3), anti-VEGF-C (n=13), anti-VEGF-A/C (n=5). Each point represents data from an individual mouse. Means±s.e.m. are also shown. **P<0.01. n.s.=not significant.

FIG. 15A is a graph showing the effect of targeting angiogenesis and/or lymphangiogenesis on the formation of regional lymph node metastases in NOD/SCID mice bearing lumen-implanted HCT116-DsRed tumors at 6-7 wpi, with the number of lymph node macroscopic metastases normalized to primary tumor volume. Data are expressed as the number of macroscopic metastases per 1 mm³ of primary tumor. Number of mice per treatment condition: untreated (n=22), anti-VEGF-A (n=9), anti-VEGF-C (n=8), anti-VEGF-NC ((n=10). Each point represents data from an individual mouse. Means±s.e.m. are also shown. *P<0.05; ***P<0.001; ****P<0.0001. n.s.=not significant.

FIG. 15B is a graph showing the effect of targeting angiogenesis and/or lymphangiogenesis on the formation of distant liver metastases in NOD/SCID mice bearing lumen-implanted HCT116-DsRed tumors at 6-7 wpi, with the number of liver macroscopic metastases normalized to primary tumor volume. Number of mice per treatment condition: untreated (n=22), anti-VEGF-A (n=9), anti-VEGF-C (n=8), anti-VEGF-A/C ((n=10). Each point represents data from an individual mouse. Means±s.e.m. are also shown. *P<0.05; ***P<0.001; ****P<0.0001. n.s.=not significant.

### DETAILED DESCRIPTION OF THE INVENTION

The present invention is based in part on the generation of a model of colorectal cancer that exhibits metastasis to clinically relevant sites.

Unless defined otherwise, technical and scientific terms used herein have the same meaning as commonly understood by one of ordinary skill in the art to which this invention belongs. Singleton et al. Dictionary of Microbiology and Molecular Biology. 2nd Ed. J. Wiley & Sons (New York, N.Y. 1994). For purposes of the present invention, the following terms are defined below.

### Definitions

The term “antibody” herein is used in the broadest sense and refers to any immunoglobulin (Ig) molecule comprising two heavy chains and two light chains, and any fragment, mutant, variant or derivation thereof so long as they exhibit the desired biological activity (e.g., epitope binding activity). Examples of antibodies include monoclonal antibodies, polyclonal antibodies, multispecific antibodies, and antibody fragments.

The term “monoclonal antibody” as used herein refers to an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to theft specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present invention may be made by the hybridoma method first described by Ohler et al., Nature. 256:495 (1975), or may be made by recombinant DNA methods (see, e.g., U.S. Pat. No. 4,816,567). The “monoclonal antibodies” may also be isolated from phage antibody libraries using the techniques described in Clackson al., Nature. 352:624-628 (1991) and Marks et al., J. Mol. Biol. 222:581-597 (1991), for example.

An “antibody fragment” refers to a molecule other than an intact antibody that comprises a portion of an intact antibody, such as the antigen-binding or variable region thereof. Examples of antibody fragments include Fab, Fab′, F(ab′)₂, and Fv fragments; diabodies; linear antibodies; single-chain antibody molecules, and multispecific antibodies formed from antibody fragment(s). in certain embodiments, the antibody fragment binds the same antigen to which the intact antibody binds.

The terms “cancer” and “cancerous” refer to or describe the physiological condition in mammals that is typically characterized by unregulated cell growth. Examples of cancer include but are not limited to, carcinoma, lymphoma, blastoma, sarcoma, and leukemia. More particular examples of such cancers include squamous cell cancer, small-cell lung cancer, non-small cell lung cancer, gastrointestinal cancer, pancreatic cancer, glioblastoma, cervical cancer, ovarian cancer, liver cancer, bladder cancer, hepatoma, breast cancer, colon cancer, colorectal cancer, endometrial carcinoma, salivary gland carcinoma, kidney cancer, renal cancer, prostate cancer, vulval cancer, thyroid cancer, hepatic carcinoma and various types of head and neck cancer.

By “metastasis” is meant the spread of cancer from its primary site to other places in the body. Cancer cells can break away from a primary tumor, penetrate into lymphatic and blood vessels, circulate through the bloodstream, and grow in a distant focus (metastasize) in normal tissues elsewhere in the body. Metastasis can be local or distant. Metastasis can be characterized as a sequential process, contingent on tumor cells breaking off from the primary tumor, traveling through the bloodstream or lymphatics, and stopping at a distant site. After the tumor cells come to rest at another site, they can re-penetrate through the blood vessels or lymphatic walls, continue to multiply, and eventually another tumor is formed. At the new site, the cells establish a blood supply and can grow to form a life-threatening mass. In certain embodiments, this new tumor is referred to as a metastatic (or secondary) tumor. In certain embodiments, the term metastatic tumor refers to a tumor that is capable of metastasizing, but has not yet metastasized to tissues or organs elsewhere in the body. In certain embodiments, the term metastatic tumor refers to a tumor that has metastasized to tissues or organs elsewhere in the body. In certain embodiments, metastatic tumors are comprised of metastatic tumor cells.

The “metastatic organ” or “metastatic tissue” is used in the broadest sense, refers to an organ or a tissue in which the cancer cells from a primary tumor or the cancer cells from another part of the body have spread. Examples of metastatic organ and metastatic tissue include, but are not limited to, lung, liver, brain, ovary, bone, bone marrow, and lymph node. With respect to colorectal cancer (CRC), predominant metastatic organ and metastatic tissue are the regional intestinal lymph nodes, liver, and lungs.

By “micrometastasis” is meant a small number of cells that have spread from the primary tumor to other parts of the body. Micrometastasis may or may not be detected in a screening or diagnostic test.

By “macrometastasis” is meant a number of cells that are detectable and have spread from the primary tumor site to other parts of the body.

By “non-metastatic” is meant a cancer that is benign or that remains at the primary site (e.g., a locally invasive cancer) and has not penetrated into the lymphatic or blood vessel system or to tissues other than the primary site. In certain embodiments, a non-metastatic cancer is any cancer that is a Stage 0, I, or II cancer,

“Invasiveness” or “invasive growth,” as used herein, refers to the ability of a cancer or tumor to leave the tissue site at which it originated and proceed to proliferate at a different site (e.g., nearby or distant site) of the body. In some embodiments, a cancer can be “locally invasive” and proceed to proliferate at a nearby site of the body, such as surrounding tissue. In other embodiments, a cancer can be “regionally invasive” or “distantly invasive” and proceed to proliferate at a regional or distant site of the body, respectively.

Reference to a cancer or tumor as a “Stage 0,” “Stage I,” “Stage II,” “Stage III,” or “Stage IV” indicates classification of the tumor or cancer using the Overall Stage Grouping or Roman Numeral Staging methods known in the art. Although the actual stage of the cancer is dependent on the type of cancer, in general, a Stage 0 cancer is an in situ lesion, a Stage I cancer is small localized tumor, a Stage II is a local advanced tumor, a Stage III cancer is a local advanced tumor that exhibits involvement of the local lymph nodes, and a Stage IV cancer represents metastatic cancer. The specific stage for each type of tumor is known to the skilled clinician.

“Tumor,” as used herein, refers to any neoplastic cell growth, whether malignant or benign, and all pre-cancerous and cancerous cells and tissues. The terms “cancer”, “cancerous”, “cell proliferative disorder”, “proliferative disorder” and “tumor” are not mutually exclusive as referred to herein. Tumors may be solid tumors, such as tumors of the colon (CRC tumor), or non-solid or soft tumors, such as leukemia. Examples of soft tissue tumors include leukemia (e.g., chronic myelogenous leukemia, acute myelogenous leukemia, adult acute lymphoblastic leukemia, acute myelogenous leukemia, mature B-cell acute lymphoblastic leukemia, chronic lymphocytic leukemia, lymphocytic leukemia, or hairy cell leukemia), or lymphoma (e.g., non-Hodgkin's lymphoma, cutaneous T-cell lymphoma, or Hodgkin's disease). A solid tumor includes any cancer of body tissues other than blood, bone marrow, or the lymphatic system. Solid tumors can be further separated into those of epithelial cell origin and those of non-epithelial cell origin. Examples of solid tumors include tumors of colon, breast, prostate, lung, kidney, liver, pancreas, ovary, head and neck, oral cavity, stomach, duodenum, small intestine, large intestine, gastrointestinal tract, anus, gall bladder, labium, nasopharynx, skin, uterus, male genital organ, urinary organs, bladder, and skin. Solid tumors of non-epithelial origin include sarcomas, brain tumors, and bone tumors. The term “tumor,” as used herein, is also meant to be inclusive of “polyps.”

By “primary tumor” or “primary cancer” is meant the original cancer and not a metastatic lesion located in another tissue, organ, or location in the subject's body. In certain embodiments, primary tumor is comprised of primary tumor cells.

By “benign tumor ” or “benign cancer” is meant a tumor that remains localized at the site of origin and does not have the capacity to infiltrate, invade, or metastasize to a distant site.

“Tumorigenic cells,” as used herein, refer to any cells (e.g., cancer cells, e.g., human cancer cells or non-human cancer cells) that exhibit an abnormal growth state or are capable of changing their normal growth state to an abnormal growth state in which they eventually form tumors. Tumorigenic cells are capable of forming tumors, which are generally the result of uncontrolled growth of the cells. Tumorigenic cells can be distinguished from non-tumorigenic cells on the basis of their tumor-forming phenotype (see, e.g., Al-Hajrj, et al. Proc Natl Acad Sci USA. 100: 3983-8, 2003; U.S. Pub. No. 2002/0119565; U.S. Pub. No. 2004/0037815; U.S. Pub. No. 2005/0232927; WO 05/005601; U.S. Pub. No. 2005/0089518; U.S. appl. Ser. No. 10/864,207; Al-Hajj et al. Oncogene. 23: 7274, 2004; and Clarke et al. Ann Ny Acad. Sci. 1044: 90, 2005, all of which are herein incorporated by reference in their entireties for all purposes). Tumorigenic cells include, without limitation, tumor cells, embryonic cells, cells engineered to have abnormal growth, cancer cell lines, as well as cell masses of any of these cell types.

The term “implant,” and variations thereof, refers to transplanted cells, for example, tumorigenic cells (e.g., an intact tumor, or fragment thereof) which are introduced into a recipient host and which remain substantially stably established at the site of transplantation in the recipient.

By “donor” cell, tumor, or tumorigenic cell is meant a cell, tumor, or tumorigenic cell that is not derived from the recipient host organism, but may be syngeneic (where the donor and recipient are genetically identical), allogeneic (where the donor and recipient are of different genetic origins but of the same species), or xenogeneic (where the donor and recipient are from different species). For example, the donor cell, tumor, or tumorigenic cell may be derived from a human. A “donor tumorigenic cell implant” refers to transplanted tumorigenic cells, as used herein, which are derived from a source other than the recipient organism.

By “tumor load” is meant the amount of cancer in the body. Tumor load is also referred to as tumor burden, and may be a function of tumor number and tumor size.

“Adjuvant therapy” herein refers to therapy given after surgery, where no evidence of residual disease can be detected, so as to reduce the risk of disease recurrence. The goal of adjuvant therapy is to prevent recurrence of the cancer, and therefore to reduce the chance of cancer-related death.

A “small molecule” is defined herein to have a molecular weight below about 500 Daltons.

By “immunoconjugate” is meant an antibody conjugated to one or more heterologous molecule(s) (e.g., an antibody-drug conjugate (ADC)), including but not limited to a cytotoxic agent.

By “reduce” or “inhibit” is meant the ability to cause an overall decrease, for example, of 20% or greater, of 50% or greater, or of 75%, 85%, 90%, 95%, or greater. In certain embodiments, reduce or inhibit can refer to the growth of tumorigenic cells or a tumor, which can be measured by a reduction or inhibition in the number of tumorigenic cells, size of tumors, tumor load, tumorigenic cell or tumor invasiveness, and/or tumor metastasis.

The term “non-human animal” refers to all animals, except humans, and includes, without limitation, birds, farm animals (e.g., cows), sport animals (e.g., horses), fish, reptiles, and non-human mammals (e.g., cats, dogs, and rodents).

The term “non-human mammal” refers to all members of the class Mammalia, except humans.

The term “rodent” refers to all members of the order Rodentia, including rats, mice, rabbits, hamsters, and guinea pigs.

### DETAILED DESCRIPTION

Colorectal cancer (CRC) initially manifests as benign polyps on the mucosal surface of the large intestine. If left unresected, these polyps can progress to invasive adenocarcinomas that penetrate through the submucosal and muscularis externa layers of the colorectal wall to reach the serosal side. Eventual regional spread to the intestinal lymph nodes and distant spread to the liver results in the outgrowth of gross metastases that are the major cause of CRC mortality. Deciphering the routes of CRC metastasis to these sites therefore has the potential to uncover therapeutic opportunities that may impact mortality rates. Investigations into metastatic routes, however, have been hampered by the lack of availability of relevant in vivo metastatic models of CRC. Indeed, despite the wide availability of xenograft, chemical-induced, and genetically-engineered models (e.g., mouse models) of CRC (Heijstek et al. Dig. Surg. 22: 16-25, 2005. Epub 2005 Apr. 14; Kobaek-Larsen et al. Comp. Med. 50 (1): 16-26, 2000; Rosenberg et al. Carcinogenesis. 30 (2): 183-196, 2009. Epub 2008 Nov. 26; Taketo et al. Gastroenterology. 136 (3): 780-798, 2009), tumors in these models fail to reproducibly metastasize to the regional intestinal lymph nodes and liver, the target organs relevant to human CRC.

Lumen Implantation Model of Colorectal Cancer

The present invention is based, at least in part, on the development of a clinically relevant model of colorectal cancer (CRC). In contrast to known models of CRC, the model of CRC of the invention is generated by a novel lumen implantation technique, and, importantly, is capable of recapitulating the etiology of human CRC.

A non-human animal (e.g., a non-human mammal) of any species, subspecies, genetic variant, tissue variant, or combination thereof, can be used in the generation of the lumen implantation model (LIM) of CRC. The non-human mammal may, for example, be a rodent. Examples of rodent species include, without limitation, rat, mouse, hamster, rabbit, guinea pig, and gerbil. The non-human mammal can be male or female. The non-human mammal can be any age, provided that the lumen implantation technique can be successfully executed. Accordingly, the non-human mammal can be, for example, less than one week old, from about one week to about five years old, from about one week to about three years old, from about two weeks to about two years old, from about three weeks to about one year old, from about four weeks to about six months old, from about six weeks to about three months old, from about eight weeks to about twelve weeks old, older than three years old, or older than five years old.

The non-human mammal can be wild-type (e.g., immune-competent) or immunodeficient. For example, when the lumen of the recipient host non-human mammal is implanted with a donor cell, tumor, or tumorigenic cell that is xenogeneic (e.g., human), the host non-human mammal is immunodeficient, When the lumen of the recipient host non-human mammal is implanted with a donor cell, tumor, or tumorigenic cell that is syngeneic, however, the host non-human mammal can be non-immunodeficient (e.g., wild-type).

In certain instances, the non-human mammal is a mouse. The mouse can be a nude mouse. The mouse can be a severely combined immunodeficient (SCID) mouse, for example, a NOD/SCID interleukin-2 receptor gamma chain null (NSG) mouse. The NSG mouse is described in Pearson et al. Curr. Top. Microbiol. Immunol. 324:25-51, 2008; Shultz et al. Curr Top Microbiol Immunol. 324:25-51 2005; Strom et al. Methods Mol. Biol. 640:491-509, 2010; McDermott et al. Blood. 116 (2): 193-200, 2010; Lepus et al. Hum. Immunol. 70 (10):790-802, 2009; Brehm et al. Clin Immunol. 135 (I):84-98, 2010. Any suitable immunodeficient non-human mammal can be used. Suitable non-human mammals include rodents, which can be obtained from such sources as The Jackson Laboratory of Bar Harbor, Me., Charles River Laboratories International, Inc. of Wilmington, Mass., and Harlan Laboratories of Indianapolis. Ind.

The lumen implantation technique involves the implantation of one or more (e.g., 1, 2, 3, 4, 5, 10, 20, 30, 40, 50, 10², 10³, 10⁴, 10⁵, 10⁶, 10⁷, 10⁸, 10⁹, or 10¹⁰ or more) donor tumorigenic cells that are capable of being implanted on the mucosal surface (luminal side) of the colon without breaching the colon wall. The donor tumorigenic cell(s) can be syngeneic (where the donor and recipient are genetically identical), allogeneic (where the donor and recipient are of different genetic origins but of the same species), or xenogeneic (where the donor and recipient are from different species, e.g., human) with respect to the recipient non-human mammal host. The donor tumorigenic cell(s) may be invasive or non-invasive, benign or malignant, metastatic or non-metastatic. The tumorigenic cells may be tumor cells, or alternatively, may be, for example, embryonic cells, cells engineered to have abnormal growth, cancer cell lines, as well as cell masses of any of these cell types.

In instances of implantation of more than one donor tumorigenic cell, the implanted cells may be an intact tumor, or fragment thereof. The intact tumor or fragment thereof, can be an intact malignant tumor, or fragment thereof, such as a Stage III CRC tumor, which has given rise to regional metastases (e.g., in the intestinal lymph node) in the donor organism, or a Stage IV CRC tumor, which has given rise to distant metastases (e.g., in the liver or lungs) in the donor organism. Alternatively, the intact tumor or fragment thereof, can be an intact benign or locally invasive tumor, or fragment thereof, such as a Stage 0, Stage I, or Stage II CRC tumor, or fragment thereof, which is confined to the site or tissue of primary origin.

The intact tumor, or fragment thereof, is not limited to a CRC tumor, or fragment thereof. For example, the intact tumor, or fragment thereof, can be an intact a non-CRC tumor, or fragment thereof, such as, without limitation, a breast cancer tumor, lung cancer tumor, liver cancer tumor, brain cancer tumor, lymph node cancer tumor, kidney cancer tumor, stomach cancer tumor, ovarian cancer tumor, skin cancer tumor, pancreatic cancer tumor, thyroid cancer tumor, or prostate cancer tumor, or fragment thereof.

The intact tumor, or fragment thereof, implanted on the colonic mucosal surface of the recipient non-human mammal host can be a solid tumor (e.g., a colon/CRC tumor, breast cancer tumor, lung cancer tumor, or liver cancer tumor), or fragment thereof. The intact tumor, or fragment thereof, can be derived from any suitable donor organism, such as a human or mouse. The intact tumor, or fragment thereof, can be from a particular cell line, such as a CRC cell line (e.g., HCT116,LS174T, or LoVo primary human CRC-derived cell line) or a breast cancer cell line (e.g., MDA-231 human breast cancer cell line).

The implanted one or more donor tumorigenic cells can be of any collective size. In instances when an intact tumor, or fragment thereof, is implanted, the intact tumor, or fragment thereof, is around 0.1-100 mm³ in size, e.g., around 1-100 mm³ in size, e.g., around 10 mm³ in size.

The implantation site can be along any region of the mucosal surface of the colon of the non-human mammal. In certain embodiments, the implantation site is located nearby the anus of the recipient non-human mammal in order to allow for the option of removal of the implanted tumor from the implantation site. In mice, for example, a tumor implantation distance of about 1-20 mm (e.g., about 5-15 mm, e.g., about 11-12.5 mm) away from the anus of the host mouse is preferable.

In general, a mouse LIM of CRC can be created by anesthetizing the mouse (e.g., by isoflurane inhalation), placing the mouse in a supine position with extremities secured and inserting a blunt-ended hemostat (Micro-Mosquito, No. 13010-12, Fine Science Tools) or other suitable tool around 1 cm into the anus, clasping a single mucosal fold (e.g., by closing the hemostat to the first notch), retracting and cleaning exteriorized mucosa (e.g, with povidone/iodine), rinsing (e.g., with lactated ringers solution), and blotting dry. One or more donor tumorigenic cells (e.g., a donor tumor fragment or intact polyp of ˜10 mm³) can be then be sutured onto the mucosa (e.g., using absorbable 4-0 vicryl sutures (Ethicon)), ensuring that the suture only penetrates the superficial mucosal layer. After rehydrating the mucosa with PBS, the exteriorized colon can be re-inserted together with the sutured tumor, thus reversing the rectal prolapsed. To minimize tumor dislodgement during defecation, mice can be housed on cage floor inserts and fed a 100% rodent liquid diet (AlN-76A, Casein Hydrolysate without Fiber; BioServe) from around 3 days pre-surgery to around 7 days post-surgery.

The generated non-human mammal LIMs of CRC have numerous advantages over established CRC models, as demonstrated in the Examples section below. These advantages of the LIM include, without limitation, the implantation of tumors onto the mucosal surface, compared to tumor implantation onto the serosal surface in the existing cecum implantation model, resulting in: (i) the potential to give rise to distant metastases in clinically relevant sites, compared to the widespread tumor dissemination throughout the peritoneal cavity due to tumor cell shedding rather than actual metastasis in the existing cecum implantation model; (ii) the ability to implant intact tumor fragments into a host mouse instead of cell suspensions that are unable to maintain tumor structure as in existing cell suspension injection models; and (iii) the maintained integrity of the colon wall compared to the likelihood of puncturing the colon wall as in existing cell suspension injection models.

Screening of Candidate Compounds that Inhibit Tumorigenesis

The LIM finds utility, for example, in the screening of candidate compounds that possess anti-cancer activity (e.g., compounds that inhibit growth of tumorigenic cells). Anti-cancer activity can include activity in directly or indirectly mediating any effect in preventing, delaying, reducing or inhibiting tumor growth and/or development, which may provide for a beneficial effect to the host. Anti-cancer activity of a candidate compound could therefore be reflected by, without limitation, the ability of the candidate compound to, directly or indirectly, reduce or stabilize: the number of tumorigenic cells (e.g., reduce the number of tumorigenic cells by at least 20%, 30%, 40%, 50%, 60%, 70%, 80%, 85%, 90%, 95%, 96%, 97%, 98%, or 99% or more), tumor size (e.g., reduce the size of a tumor by at least 20%, 30%, 40%, 50%, 60%, 70%, 80%, 85%, 90%, 95%, 96%, 97%, 98%, or 99% or more), tumor load or burden (e.g., reduce tumor load or burden by at least 20%, 30%, 40%, 50%, 60%, 70%, 80%, 85%, 90%, 95%, 96%, 97%, 98%, or 99% or more), tumorigenic cell invasiveness (e.g., reduce invasiveness nearby tissue by at least 20%, 30%, 40%, 50%, 60%, 70%, 80%, 85%, 90%, 95%, 96%, 97%, 98%, or 99% or more), and/or tumor metastasis (e.g., reduce the number of metastases and/or metastatic organs or tissues by at least 20%, 30%, 40%, 50%, 60%, 70%, 80%, 85%, 90%, 95%, 96%, 97%, 98%, or 99% or more). Accordingly, the anti-cancer activity of a candidate compound can be assessed by determining the presence or absence of, for example, one or more of the above effects related to the inhibition of growth of tumorigenic cells in the LIM, wherein the presence of one or more effects on tumorigenic cell growth is indicative of the candidate compound possessing anti-cancer activity. For example, with respect to determining an effect on tumor size and/or number, the determining step can include measuring tumor size and/or number at a first time point and a second time point, comparing tumor size and/or number measured at the first time point relative to that measured at the first time point. In some instances, with respect to tumor invasiveness and metastasis, the determining step can include detecting the presence or absence of tumor invasion (e.g., invasive tumor growth through the colon wall to the colonic serosal surface) or metastases (e.g., metastases in the intestinal lymph nodes, liver, and/or lungs) by gross visual analysis (e.g., when detecting macrometastases) and/or by histological or cell counting analyses.

The candidate compounds that can be screened for anti-cancer activity using a LIM of the present invention include, without limitation, synthetic, naturally occurring, or recombinantly produced molecules, including small molecules, polynucleotides, peptides, polypeptides, antibodies, and immunoconjugates. Candidate compounds can be obtained from a wide variety of sources including libraries of synthetic or natural compounds. For example, numerous means are available for random and directed synthesis of a wide variety of organic compounds and biomolecules, including expression of randomized oligonucleotides and oligopeptides. Alternatively, libraries of natural compounds in the form of bacterial, fungal, plant, and animal extracts are available or readily produced. Additionally, natural or synthetically produced libraries and compounds are readily modified through conventional chemical, physical, and biochemical means, and may be used to produce combinatorial libraries. Known pharmacological agents may be subjected to directed or random chemical modifications, such as acylation, alkylation, esterification, and amidification, to produce structural analogs.

The candidate compounds may be formulated, dosed, and administered in any manner desired and/or appropriate in a fashion consistent with good medical practice and in order to examine anti-cancer activity. The candidate compounds may be prepared in therapeutic formulations using standard methods known in the art by mixing the active ingredient having the desired degree of purity with optional physiologically acceptable carriers, excipients or stabilizers (Remington's Pharmaceutical Sciences (20^{th} edition), ed. A. Gennaro, 2000, Lippincott, Williams & Wilkins, Philadelphia, Pa.). Acceptable carriers, include saline, or buffers such as phosphate, citrate and other organic acids; antioxidants including ascorbic acid; low molecular weight (less than about 10 residues) polypeptides; proteins, such as serum albumin, gelatin or immunoglobulins; hydrophilic polymers such as polyvinylpyrrolidone, amino acids such as glycine, glutamine, asparagines, arginine or lysine; monosaccharides, disaccharides, and other carbohydrates including glucose, mannose, or dextrins; chelating agents such as EDTA; sugar alcohols such as mannitol or sorbitol; salt-forming counterions such as sodium; and/or nonionic surfactants such as TWEEN™, PLURONICS™, or PEG.

Optionally, the formulation contains a pharmaceutically acceptable salt (e.g., sodium chloride) at about physiological concentrations. Optionally, the formulations of the invention can contain a pharmaceutically acceptable preservative. In some embodiments the preservative concentration ranges from 0.1 to 2.0%, typically v/v. Suitable preservatives include those known in the pharmaceutical arts. Benzyl alcohol, phenol, m-cresol, methylparaben, and propylparaben are preferred preservatives. Optionally, the formulations of the invention can include a pharmaceutically acceptable surfactant at a concentration of 0.005 to 0.02%.

The candidate compounds can be administered singly or can be combined in combinations of two or more (e.g., 3, 4, or 5 or more candidate compounds), especially where administration of a combination of compounds may result in a synergistic effect.

Adjuvant Model of Colorectal Cancer and Screening of Adjuvants

The LIM can also be utilized to generate an adjuvant model of CRC to be subsequently used, for example, in the screening of adjuvants that possess anti-cancer activity (e.g., compounds that inhibit of growth of tumorigenic cells). To this end, the implanted primary tumor is surgically removed after implantation, and adjuvant screening with candidate compounds can then be performed on the non-human mammal (e.g., rodent, e.g., mouse or rat) adjuvant model in a manner analogous to the screening of compounds that inhibit growth of tumorigenesis, discussed above.

In this adjuvant model of colorectal cancer, the surgical removal of the implanted primary tumor can be performed at various time points post-implantation, corresponding to different stages of CRC disease progression (e.g., Stage 0, I, II, III, or IV). The same or different candidate compounds can then be tested for efficacy as an adjuvant in the treatment of different stages of CRC.

A candidate compound that inhibits growth/re-growth of tumorigenic cells in an adjuvant setting compared to a counterpart untreated or control-treated adjuvant model identifies a candidate compound as an adjuvant.

The duration of adjuvant therapy trials, as well as the formulation, dosage, and administration route of an adjuvant candidate or identified adjuvant can be altered as necessary in any manner desired and/or appropriate in a fashion consistent with good medical practice, similar to candidate compounds for primary therapy, as described above.

### EXAMPLES

The present invention is illustrated by the following Examples, which are in no way intended to be limiting of the invention.

### Example 1

### Materials and Methods

One skilled in the art will recognize many materials and methods similar or equivalent to those described herein, which could be used in the practice of the present invention. Indeed, the present invention is in no way limited to the materials and methods described below.

Mice

Wild-type NOD/SCID female mice (8-12 weeks old) were purchased from Charles River Laboratories. Wild-type NSG female mice (8-12 weeks old; stock number 00 55 57), Apc^{Min/+} mice (stock number 002020), and 12.4 KbVilCre mice (stock number 004586; referred to as Villin-Cre) were purchased from the Jackson Laboratory. Kras^{LSLG12D/+} mice were licensed from Tyler Jacks from the Massachusetts Institute of Technology. Apc/Kras compound mutant mice from colony number 4028 were bred with CAG-mRFP1 mice (stock number 005884) purchased from the Jackson Laboratory. Apc/Kras compound mutant mice from colony number 4700 were bred with Rosa26-CAG-LSL-tdTomato mice (stock number 007909) purchased from the Jackson Laboratory. All experiments were approved by the Animal Research Ethics and Protocol Review Committee of Genentech.

Cell Culture and Gene Transfer

HCT116,LS174T, and LoVo primary human colorectal cancer-derived cell lines were purchased from ATCC and maintained in complete RPMI medium (RPMI 1640 supplemented with 10% fetal bovine serum, 2 mM glutamine, 100 U/ml penicillin, and 100 mg/ml streptomycin) at 37° C. and 5% CO₂. Cells were transduced with a TZV-CMV-Discosoma red fluorescent protein (DsRed) lentiviral vector (Open Biosystems) at a multiplicity-of-infection (MOI) of 10 in complete RPMI medium supplemented with 8 mg ml⁻¹ polybrene for 6 hr at 37° C. and 5% CO₂. After 4 passages in culture, DsRed-positive cells were isolated by fluorescence-activated cell sorting on a FACSAria (BO Biosciences). Sorted DsRed-positive cells were expanded for 2-3 passages, and then stored in liquid nitrogen. Early passage cells were used for all in vivo experiments.

Lumen Implantation Technique

Mice were anesthetized by isoflurane inhalation, placed in a supine position, and the extremities secured to a gauze-covered platform with tape. A blunt-ended hemostat (Micro-Mosquito. No. 13010-12, Fine Science Tools) was inserted ˜1 cm into the anus, and the hemostat angled towards the mucosa and opened slightly such that a single mucosal fold could be clasped by closing the hemostat to the first notch. The hemostat was retracted from the anus, and the clasped exteriorized mucosa cleansed with povidone/iodine, rinsed with lactated ringers solution and blotted dry. A donor tumor fragment or intact polyp of ˜10 mm³ was sutured onto the mucosa using absorbable 4-0 vicryl sutures (Ethicon), ensuring that the suture only penetrated the superficial mucosal layer. After rehydrating the mucosa with PBS, the hemostat was released and a blunt gavage needle used to re-insert the exteriorized colon together with the sutured tumor, thus reversing the rectal prolapse. The average tumor implantation distance was 11.8±0.5 mm away from the anus (n=18). Mortality post-surgery was less than 1%, with morbidity at 2-4 wpi attributable to reversible rectal prolapse in less than 5% of mice and morbidity at 7 wpi attributable to weight loss due to increased tumor burden. To minimize tumor dislodgement during defecation, mice were housed on cage floor inserts and fed a 100% rodent liquid diet (AlN-76A, Casein Hydrolysate without Fiber; BioServe) from 3 days pre-surgery until 7 days post-surgery.

Subcutaneous Tumor Generation

Cell lines were harvested via trypsinization, counted with trypan blue to assess viability, and resuspended in cold complete RPMI medium at a concentration of 100×10⁶ cells/ml. Cold Matrigel (BD Biosciences) was added to the cell suspension at a 1:1 ratio to achieve a final cell concentration of 50×10⁶ cells/ml. NOD/SCID mice were injected with 5×10⁶ cells in a volume of 100 μl subcutaneously in the left flank. Tumor dimensions were measured using calipers and tumor volume was calculated as 0.523×length×width×width. For subcutaneous tumors used as donors for the lumen implantation technique, tumors were harvested between 1000-2000 mm³, necrotic tissue grossly dissected away under a microscope, and the remaining viable tissue divided into 10 mm³ fragments and placed on ice in complete RPMI medium.

Endoscopy

Prior to endoscopic imaging, mice were anesthetized by isoflurane inhalation, placed in a supine position, and their colons evacuated of stool using a gavage needle. Endoscopic imaging equipment consisted of a Hopkins II 0° straight forward 1.9 mm outer diameter telescope encompassed by an examination and protection sheath, an Image-I high definition three-chip digital camera attached to a Mikata Point Setter telescope holding system, a fiber optic light guide cable connected to a D Light System xenon light source, an electronic CO₂ insufflator to maintain colon insufflation during imaging, and an AIDA Connect high definition documentation system connected to a high definition color monitor (Karl Storz). Endoscopic videos were reviewed using VLC Media Player (VideoLAN Team) and still images were captured from these videos.

Whole Organ Imaging and Macrometastasis Evaluation

Colons were harvested intact, flushed with PBS, opened longitudinally, pinned down on thin cardboard pieces, and imaged both mucosally and serosally. Livers and lungs were harvested, washed in PBS, and imaged. All organs were imaged using a DFC295 color digital camera (Leica) attached to a M80 stereomicroscope (Leica). Macroscopic metastasis formation was assessed visually using a S4 stereomicroscope (Leica). For the intestinal lymph nodes, the entire intestinal tract from the anus to the stomach was examined for evidence of lymph node involvement, and the number of macrometastases quantified. For the liver and lungs, the entire external surface of whole organs was examined and the number of macrometastases quantified. Following macro metastasis quantification, organs were fixed in 4% paraformaldehyde in PBS overnight. Prior to overnight fixation in 4% paraformaldehyde, lungs were perfused with 4% paraformaldehyde in PBS. Primary colorectal tumor dimensions were determined using a reference measurement scale and tumor volume was calculated as 0.523×length×width×width.

Tissue Digestion and Flow Cytometry

Entire tissues were processed on a GentieMACS dissociator (Miltenyi Biotec), digested in complete RPMI medium supplemented with 1 mg/ml collagenase/dispase for 30 min at 37° C. with agitation at 210 rpm, and filtered through a 70-μm strainer. Following red blood cell lysis and centrifugation, cells were resuspended in PBS supplemented with 2% fetal bovine serum, 20 mM HEPES and 5 μg/ml propidium iodide, filtered into FACS tubes and analyzed on a FACSAria flow cytometer (BO Biosystems). For FACS controls, normal tissues from non-tumor-bearing mice were used, and DsRed-positive analysis gates were established such that zero DsRed-positive events were detectable in control tissue specimens. An average of 5×10⁶ viable events were analyzed per specimen. Data were expressed as the number of DsRed-positive cells per 1×10⁶ viable events.

Circulating Tumor Cells

Mice were euthanized by CO₂ inhalation. Immediately after breathing subsided, the rib cage was splayed open to expose the heart. A syringe fitted with a 27 gauge needle was inserted into the right chamber of the heart, and ˜50 μl of blood was withdrawn. Blood was immediately transferred to EOTA-coated Microtainer tubes (BD Biosciences). Following red blood cell lysis, blood samples were resuspended in PBS supplemented with 2% fetal bovine serum, 20 mM HEPES, and 5 μg/ml propidium iodide, and analyzed by flow cytometry. For FACS controls, blood from non-tumor-bearing mice was used, and DsRed-positive analysis gates were established such that zero DsRed-positive events were detectable in control blood specimens. An average of 5×10⁶ viable events were analyzed per specimen. Data were expressed as the number of DsRed-positive cells per 1×10⁶ viable events.

Human Colorectal Cancer Clinical Specimens

Freshly resected human colorectal cancer specimens were obtained from Bio-options Inc., from consenting patients in accordance with federal and state guidelines. Specimens were shipped overnight at 4° C. in DMEM high glucose medium supplemented with 10% fetal bovine serum, glutamine, vancomycin, metronidazole, cefotaxime, amphotericin B, penicillin, streptomycin, and protease inhibitor cocktail. Specimens were cut into ˜2 mm³ tumor fragments, and individual fragments implanted under the kidney capsule of athymic nu/nu male mice (6-8 weeks old) purchased from Harlan Sprague Dawley. Six months post-implantation, tumors that grew under the kidney capsule were used as donor tumors, and ˜2 mm³ donor tumor fragments were implanted into the colonic lumens of NOD/SCID mice.

Anti-VEGF-A and Anti-VEGF-C Antibodies

The anti-VEGF-A monoclonal antibody G6-31 has been described previously (U.S. Pat. No. 7,758,859; Liang et al. J. Biol. Chem. 281 (2): 951-961, 2006. Epub 2005 Nov. 7). The anti-VEGF-C monoclonal antibody VC4.5 was isolated from synthetic phage antibody libraries built on a single framework (Lee et al. J. Mol. Biol. 340: 1073-1093, 2004) by selection against a matured form of human VEGF-C (R&D Systems). One positive clone VC4 as full-length IgG was verified to block the interaction between human VEGF-C and human VEGFR3, inhibit VEGF-C induced cell activity and cross-bind murine VEGF-C. VC4 was further affinity improved to VC4.5 with phage display selection, as previously described (Lee et al. Blood. 108: 3103-3111, 2006. Epub 2006 Jul. 13) and shown to improve the potency of blocking VEGF-C from receptor binding and cell signaling. VC4.5 exhibits similar affinity towards human and murine VEGF-C (Kd=0.3-1 nM) as determined by surface plasmon resonance measurement using BIAcore instruments by immobilizing either VC4.5 IgG or VEGF-C on the chip. If desired, other anti-VEGF-A or anti-VEGF-C antibodies may be utilized.

Vascular Targeting

One day prior to lumen implantation, NOD/SCID mice were treated with the function-blocking monoclonal antibodies anti-VEGF-A (G6-31; 5 mg/kg in PBS) and/or anti-VEGF-C (VC4.5; 40 mg/kg in PBS) by intraperitoneal injection. On day 0, HCT116-DsRed tumor fragments were implanted onto the colonic mucosa. Antibodies were administered once per week.

Histopathology and Immunostaining

Tissues were fixed in 4% paraformaldehyde in PBS overnight, rinsed in PBS, cryoprotected in 30% sucrose in PBS overnight at 4° C., embedded in Optimal cutting temperature (OCT) compound and frozen at −80° C., and sectioned at 8 μm. For histopathological analyses, tissue sections were stained with haematoxylin and eosin (H&E) using a Jung Autostainer XL (Leica), and whole tissue section scans were acquired using a NanoZoomer (Hamamatsu). For immunohistochemical analyses, tissue sections were incubated with primary antibody overnight at 4° C. and secondary antibody for 30 min at room temperature. Primary antibodies used were rabbit anti-collagen IV (polyclonal ab6586; Abcam; 1:100 dilution), goat anti-DsRed (polyclonal sc-33354; Santa Cruz Biotechnology; 1:100 dilution), and rat anti-pan endothelial cell marker (clone MECA-32; Pharmingen; 2 μg/ml). Secondary antibodies used were conjugated to Alexa Fluor 488 or 594 (Invitrogen). Images were acquired on an Axioplan 2 imaging microscope (Zeiss) with an ORCA-ER digital camera (Hamamatsu). Vascular density was expressed as a ratio of the MECA-32-positive vascular area over the total DAPI-positive viable tumor area multiplied by 100. Histology specimens were reviewed by a trained pathologist with CRC disease expertise.

Statistical Analyses

Group differences were evaluated by two-tailed Student's t test. Correlations were evaluated by Pearson correlation coefficients. Contingency analyses were evaluated by two-sided chi-squared test, using actual mouse numbers as input data. For Kaplan-Meier survival analyses, P values were computed using the Log-rank test, and hazard ratios were computed using Apc^{min/+}; Villin-Cre mice as the comparator. P values less than 0.05 were considered significant.

### Example 2

### Development of a Clinically Relevant Lumen Implantation Model of Colorectal Cancer

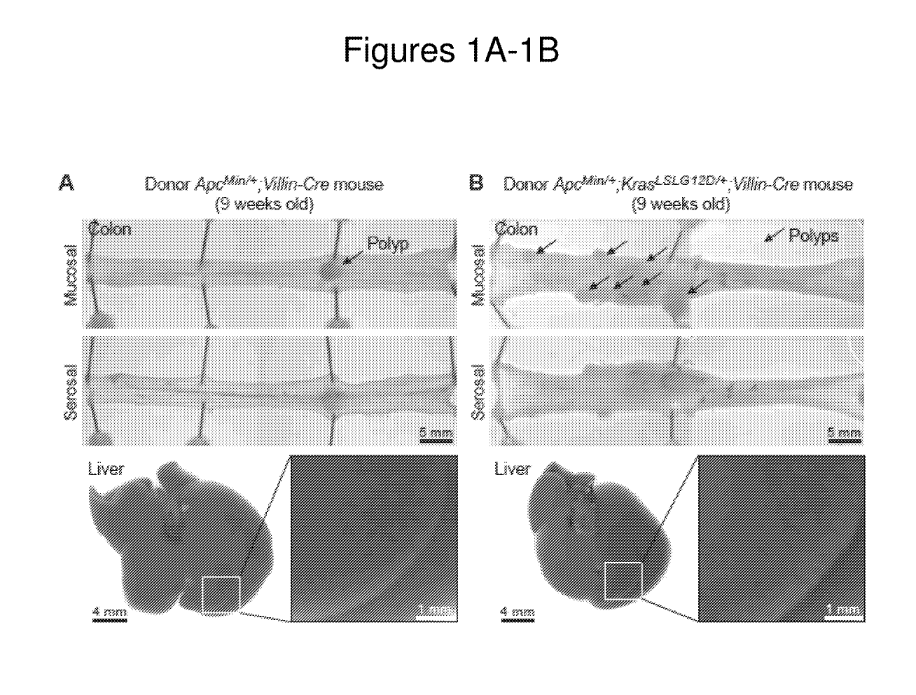

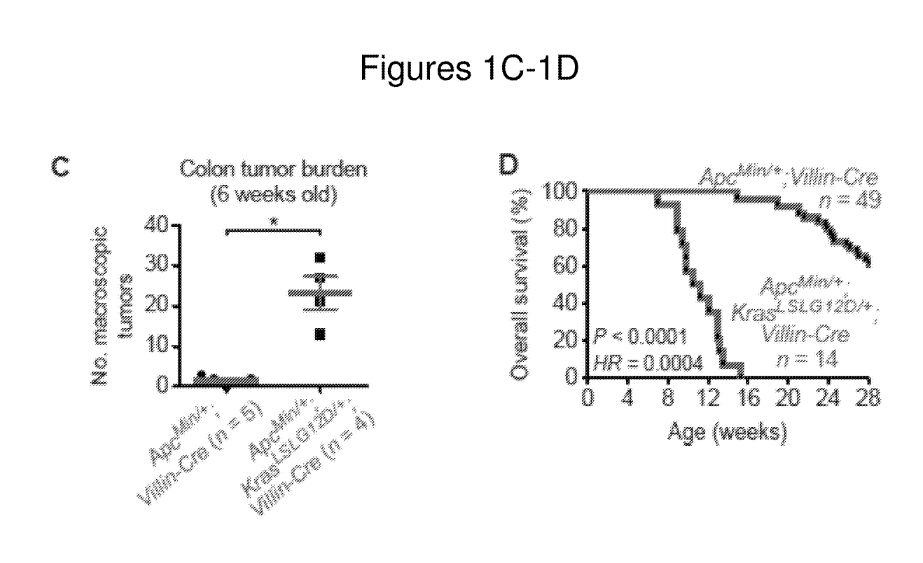

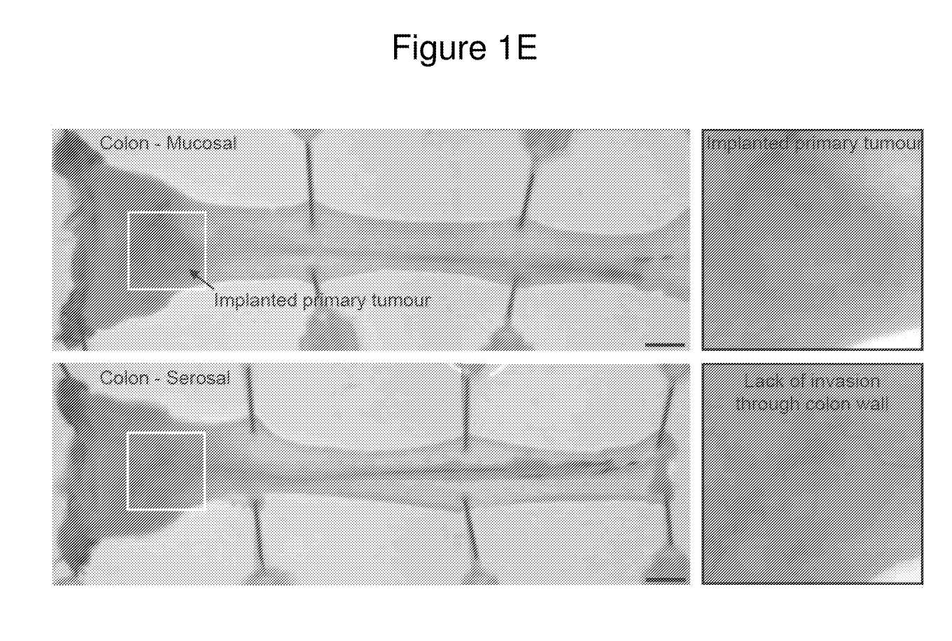

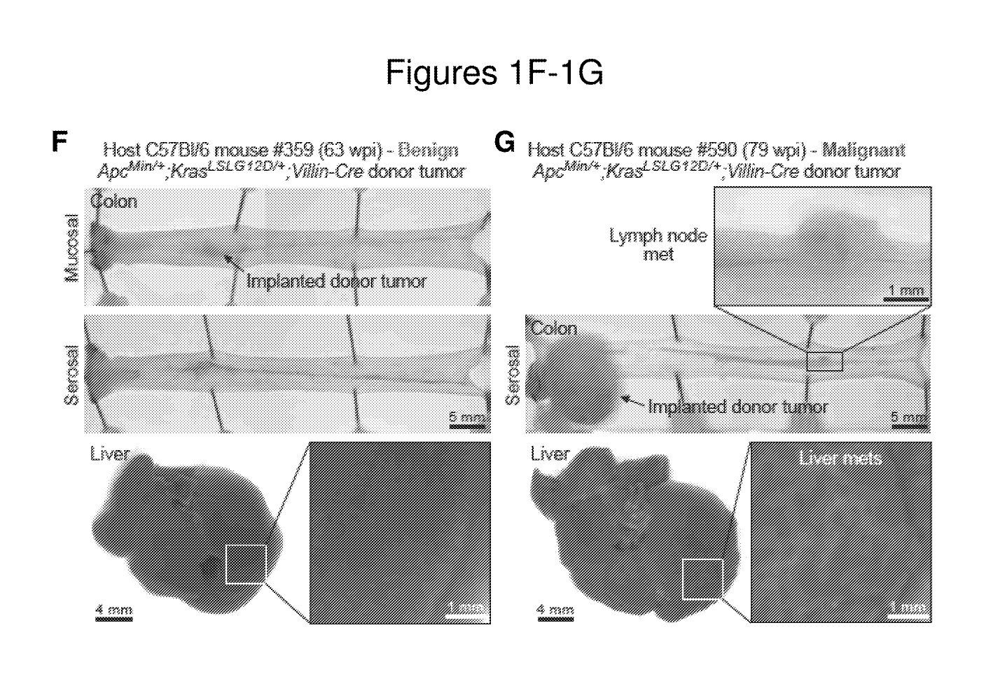

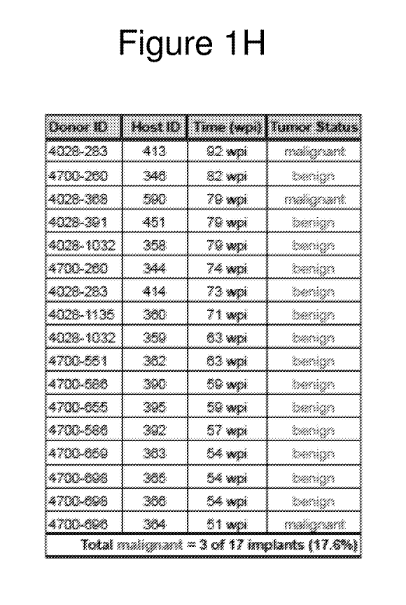

The most widely utilized genetically-engineered mouse model of intestinal cancer is the Apc^{Min/+} mouse, which harbors a dominant nonsense mutation in one Apc allele (Su at al. Science. 256 (5057): 668-670, 1992). Apc^{Min/+} mice develop numerous adenomas within the intestinal tract; however, these adenomas rarely, if ever, progress to invasive or metastatic adenocarcinomas (Moser et al. Science. 247 (4940): 322-324, 1990). Moreover, these adenomas primarily localize to the small intestine, with relatively few adenomas manifesting in the colon (Moser at al. Science. 247 (4940): 322-324, 1990). Introduction of oncogenic Kras to the mutant Apc background promotes intestinal adenoma multiplicity (Janssen at al. Gastroenterology. 131 (4): 1096-1109, 2006. Epub 2006 Aug. 16; Luo et al. Int. J. Exp. Pathol. 90 (5): 558-574, 2009) and accelerates progression to invasiveness (Janssen et al. Gastroenterology. 131 (4): 1096-1109, 2006. Epub 2006 Aug. 16; Haigis et al. Nat. Genet. 40 (5): 600-608, 2008. Epub 2008 Mar. 30; Sansom et al. Proc. Natl. Acad. Sci. USA. 103 (38): 14122-14127, 2006. Epub 2006 Sep. 7), with a marked enhancement of tumor development in the relevant anatomical location of the colon (Luo et al. Int. J. Exp. Pathol. 90 (5): 558-574, 2009). Development of intestinal lymph node metastases has not been observed in Apc/Kras compound mutant mice, with distant liver metastases only detected in 20-27% of Apc/Kras compound mutant mice by transgene RT-PCR (Janssen at al. Gastroenterology. 131 (4): 1096-1109, 2006. Epub 2006 Aug. 16) or gross observation (Hung et al. Proc. Natl. Acad. Sci. USA. 107: 1565-1570, 2010). We generated Apc^{Min/+}; Kras^{LSLG12D/+}; Villin-Cre compound mutant mice (mice carrying a Cre-dependent activated allele of Kras (Kras^{LSLG12D}) on the Ape^{Min/+} background, crossed with mice carrying a Villin-Cre transgene that directs expression of Cre recombinase throughout the intestine), and confirmed an enhancement of tumor development in the colon compared to Apc^{Min/+}; Villin-Cre control mice (FIGS. 1A-1C). As a consequence of accelerated intestinal tumorigenesis, however, Apc^{Min/+}; Kras^{LSLG12D/+}; Villin-Cre mice exhibited a dramatically reduced lifespan (FIG. 1D), consistent with previous reports (Luo et al. Int. J. Exp. Pathol. 90 (5): 558-574, 2009; Haigis et al. Nat. Genet. 40 (5): 600-608, 2008. Epub 2008 Mar. 30). Of note, disease status at the onset of morbidity at approximately 9 weeks of age remained benign, as evidenced by primary colon tumors that failed to invade through to the serosal side of the colon wall and a lack of metastasis formation in the liver (FIG. 1B).

Given that Apc/Kras compound mutant tumors exhibit features of early stage malignant progression (Janssen et al. Gastroenterology. 131 (4): 1096-1109, 2006. Epub 2006 Aug. 16; Haigis et al. Nat. Genet. 40 (5): 600-608, 2008. Epub 2008 Mar. 30; Sansom et al. Proc. Natl. Acad. Sci. USA. 103 (38): 14122-14127, 2006. Epub 2006 Sep. 7), we postulated that maintaining a compound mutant tumor in vivo beyond the shortened lifespan of an Apc/Kras mutant mouse might enable a realization of metastatic potential. We therefore aimed to transplant a single intact Ape^{Min/+}; Kras^{LSLG12D/+}; Villin-Cre donor tumor within the mucosal layer of a host wild-type C57BL/6 mouse colon, as this would faithfully represent an orthotopic primary colorectal tumor at clinical stage 0 with the potential to progress to stage IV disease. Several colon orthotopic transplantation techniques have been described to date, including the injection of cancer cell suspensions directly into the rectal mucosa (Donigan et al. Surg. Endosc. 24 (3): 642-647, 2010. Epub 2009 Aug. 18) or serosal wall of the cecum (Cespedes et al. Am. J. Pathol. 170 (3): 1077-1085, 2007), the instillation of tumor cell suspensions into the colonic lumen following electrocoagulation of the mucosa to promote tumor cell uptake (Bhullar et al. J. Am. Call. Surg. 213 (1): 54-60; discussion 60-61, 2011. Epub 2011 Mar. 31), and the surgical implantation of intact tumor fragments onto the serosal side of the cecal wall (Fu et al. Natl. Acad. Sci. USA. 88 (20): 9345-9349, 1991; Jin et al. Tumour. Biol. 32 (2): 391-397, 2011. Epub 2010 Nov. 19). One drawback of the cecum implant procedure is the fact that tumors are implanted on the serosal side of the cecal wall, thus bypassing the requirement of primary tumor invasion through the mucosa to the serosa for metastasis to occur. Importantly, a major caveat of all of these established techniques is the potential for inadvertent seeding of tumor cells into the peritoneal space, whether from a breach of the colon wall during tumor cell injection into the mucosa, or from the shedding of tumor cells from the cecal implant following return to the peritoneal cavity. Indeed, although these techniques have been utilized to generate mouse models of colorectal cancer (CRC) that reportedly develop metastases in the intestinal lymph nodes, liver and lungs, these techniques also give rise to widespread peritoneal carcinomatosis (Bhullar et al. J. Am. Call. Surg. 213 (1): 54-60; discussion 60-61, 2011. Epub 2011 Mar. 31; Cespedes et al. Am. J. Pathol. 170 (3): 1077-1085, 2007; Fu et al. Natl. Acad. Sci. USA. 88 (20): 9345-9349, 1991; Jin et al. Tumour. Biol. 32 (2): 391-397, 2011. Epub 2010 Nov. 19). It is thus plausible that tumor formation in these secondary sites may not be true metastases, but rather a result of peritoneal seeding. That peritoneal carcinomatosis is not a prominent feature of human metastatic CRC (Klaver et al. World. J. Gastroenterl. 18 (39): 5489-5494, 2012) further suggests a limited utility of these models for investigating routes of metastatic dissemination.

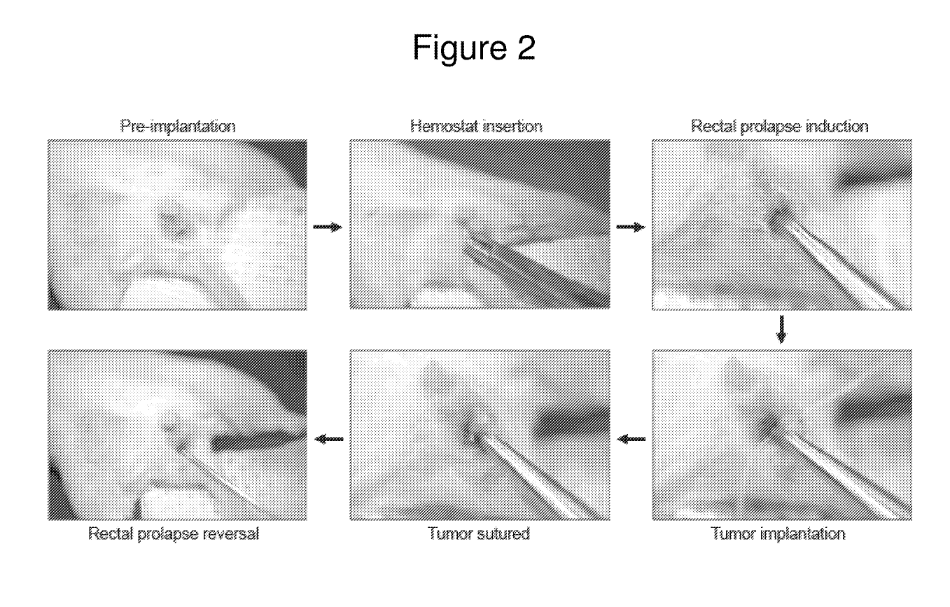

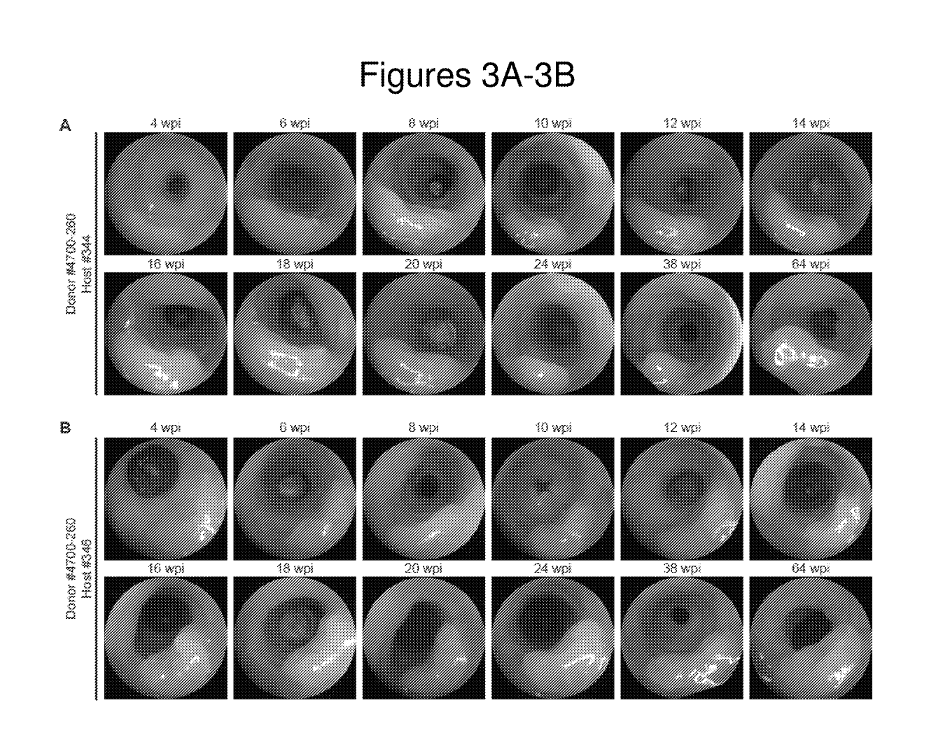

To circumvent these issues, we have developed a rectal prolapse induction technique that exteriorizes the lumen of the host colon, thus rendering the colonic mucosal surface amenable to surgical manipulation (FIG. 2). Using 6-9 week old Apc^{Min/+}; Kras^{LSLG12D/+}; Villin-Cre mice as donors, we surgically implanted single intact donor colon tumors into wild-type C57BI/6 host colons. Of note, donor tumors were not dissociated to single cells prior to implantation, but rather were maintained as intact tumor tissues as this would preserve tumor cell-stromal cell interactions, as well as extracellular matrix interactions. These intact donor tumors became established within the mucosal layer and exhibited long-term persistence in vivo as evidenced by serial endoscopy (FIGS. 3A and 3B) and gross colon biopsy at 9 wpi (FIG. 1E). At host mouse sacrifice due to age-related morbidity, whereas the vast majority of implanted donor tumors remained benign (FIGS. 1F, 3A, and 3B), a subset of mice exhibited malignant progression with donor tumor invasion through the colon wall and concomitant intestinal lymph node and liver metastasis formation (FIG. 1G). Given that malignant progression was observed in only 3 of 17 (17.6%) host mice at implantation times of 51-92 weeks post-implantation (wpi; FIG. 1H), our data support a requirement for additional genetic, epigenetic, and/or microenvironmental changes over a prolonged time period for tumor progression and metastasis to occur.

### Example 3

### Routes of Metastatic Dissemination Using the Lumen Implantation Model of CRC

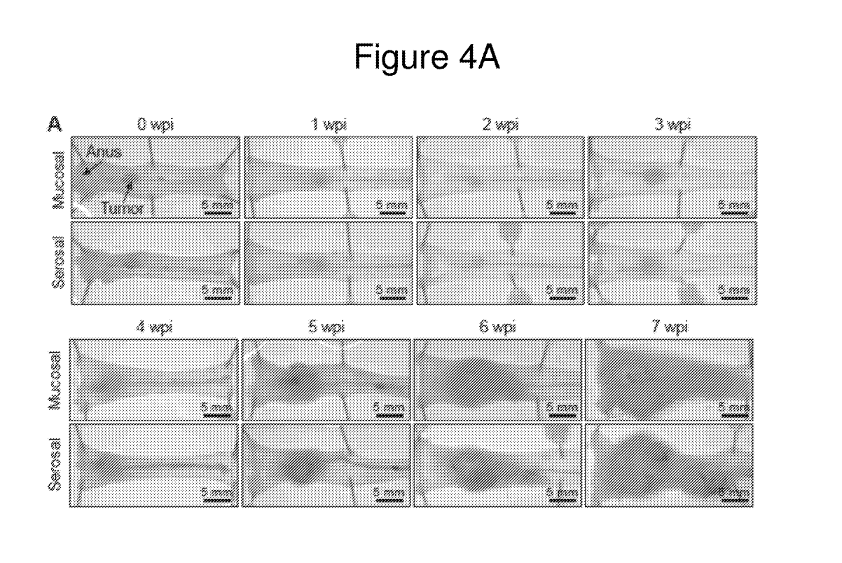

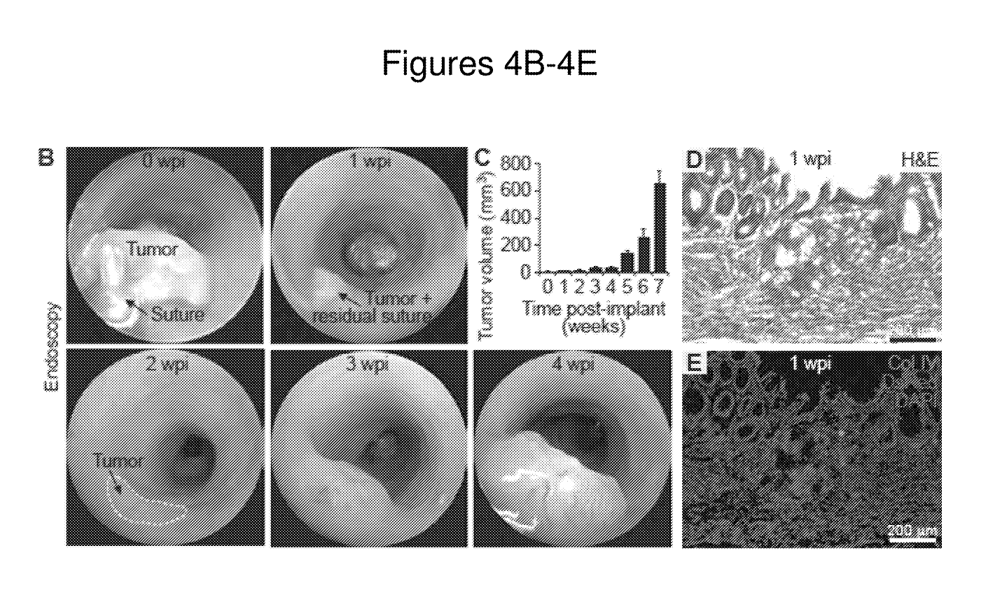

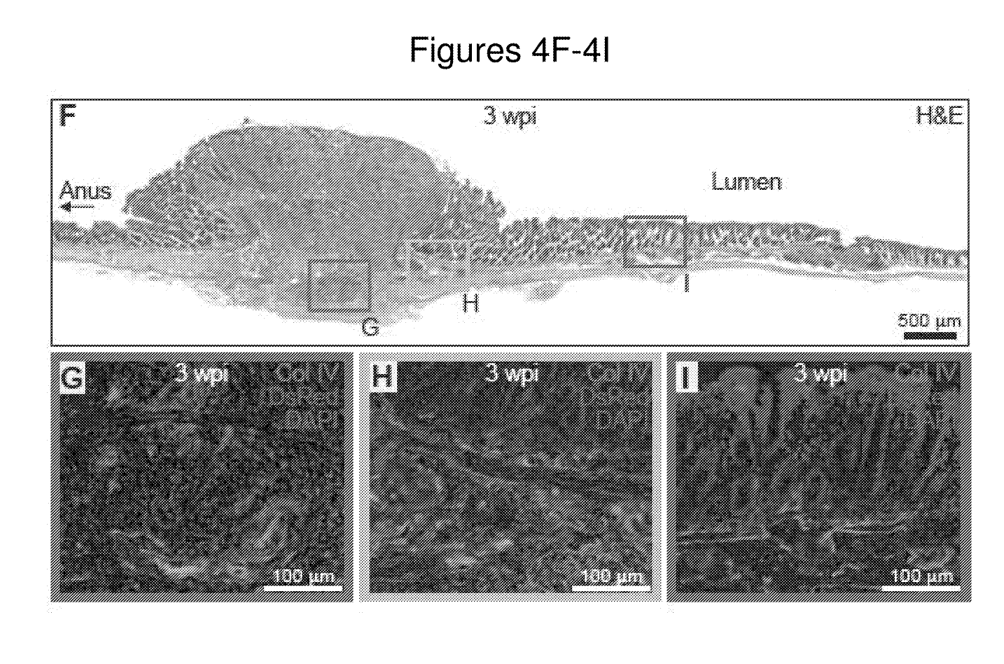

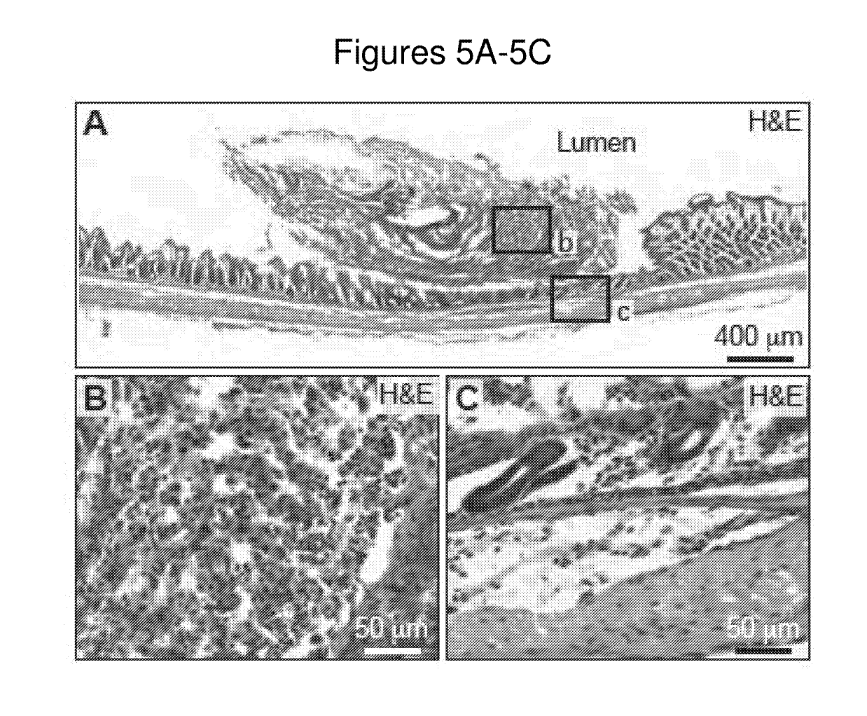

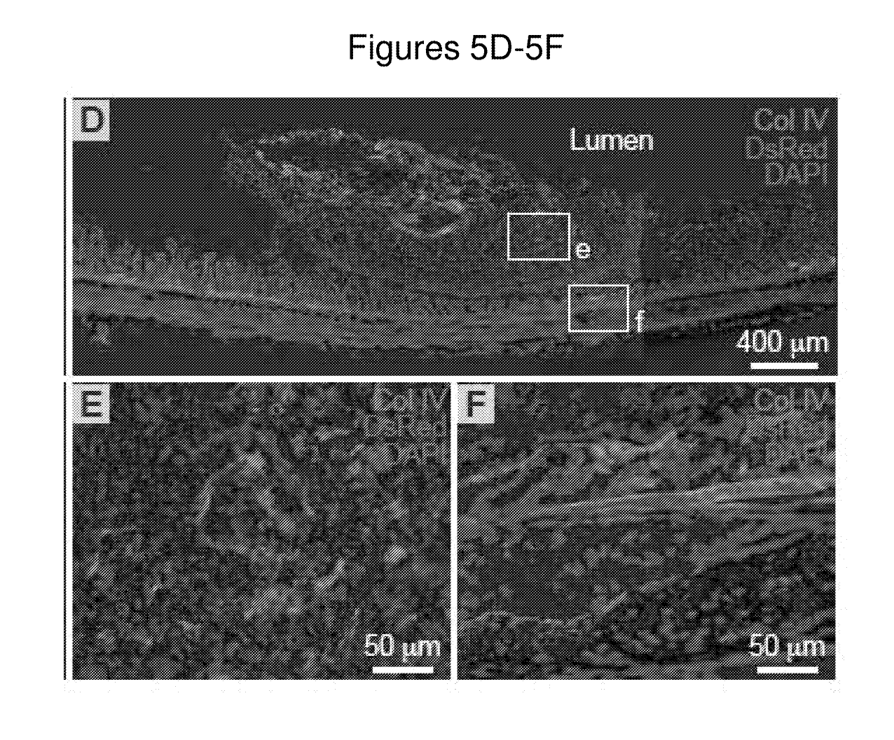

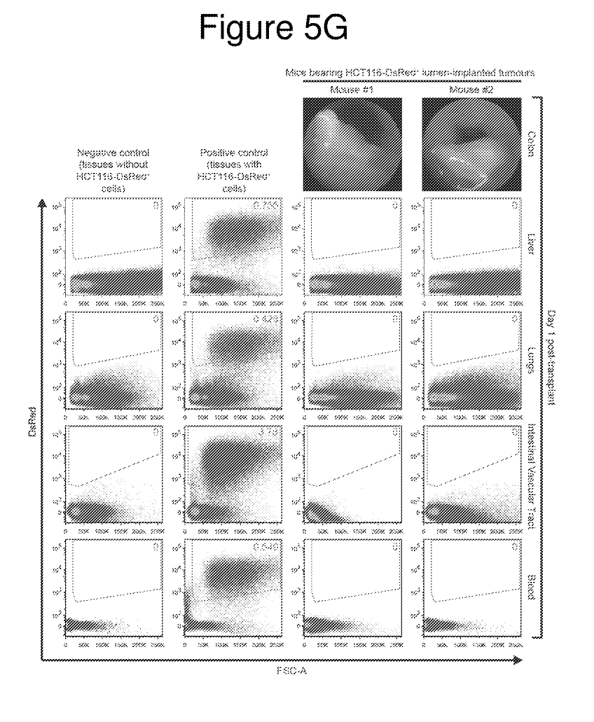

In an effort to shorten the time frame of malignant progression in our model so that routes of metastasis could be interrogated, we applied our lumen implantation technique to the poorly-differentiated HCT116 human CRC-derived cell line. HCT116 cells were transduced with the gene encoding the red fluorescent protein, DsRed, and implanted subcutaneously in a mouse to generate donor xenograft tumors. Following surgical implantation of donor tumor fragments of ˜10 mm³ onto the mucosal surface of host NOD/SCID mouse colons, ex vivo gross imaging (FIG. 4A) and in vivo endoscopy (FIG. 4B) were used to monitor tumor take rate and growth over time (FIG. 4C). Implanted tumors initially established and grew within the luminal space of the colon (FIGS. 4A and 4B), with single tumor foci detectable as intramucosal carcinomas 1 wpi (FIGS. 4D and 4E). Histological assessment immediately post-implantation confirmed that the implantation procedure did not breach the thickness of the colon wall, as primary tumors were localized exclusively on the mucosal surface of the colon (FIGS. 5A, 5B, 5D, and 5E) with the integrity of the deeper wall layers maintained (FIGS. 5C and 5F). There was also no evidence of tumor cell dissemination at Day 1 post-transplantation, as assessed by flow cytometry (FIG. 5G).

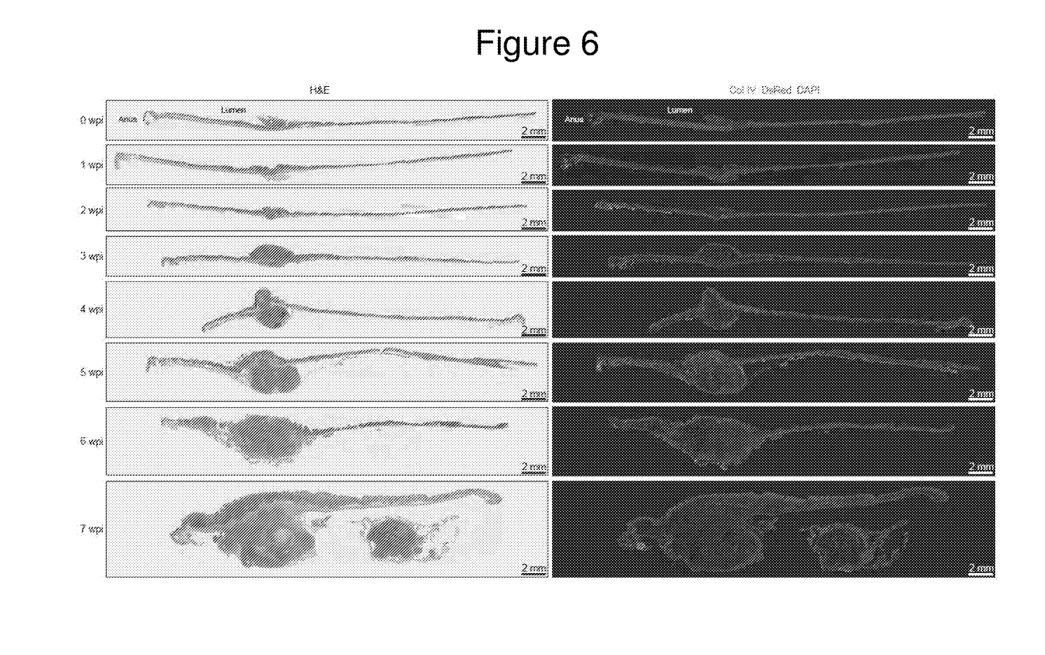

Stage 0 polyps invariably progressed to stage I tumors, which breached the submucosal/muscularis externa layers, such that by 2-3 wpi invasive stage II adenocarcinomas that penetrated through the collagen IV-rich basement membrane of the muscularis externa (Vreemann et al. Biol. Chem. 390: 481-492, 2009) to reach the serosal side of the colon wall were evident (FIGS. 4A and 6). Of note, at 3 wpi individual clusters of invasive tumor cells were detectable within the colon wall both adjacent to (FIGS. 4F-4H) and distant from (FIGS. 4F and 4I) the primary adenocarcinoma. Continued expansion of the primary adenocarcinoma occurred predominantly on the serosal side of the colon wall (FIGS. 4A and 6), to the extent that luminal obstruction was not evident even at the harvest endpoint of 7 wpi. Rather, animal morbidity at 7 wpi was attributable primarily to weight loss due to primary tumor burden, a common symptom also observed in CRC patients with later stage disease (Jellema et al. BMJ. 340: 1269, 2010).

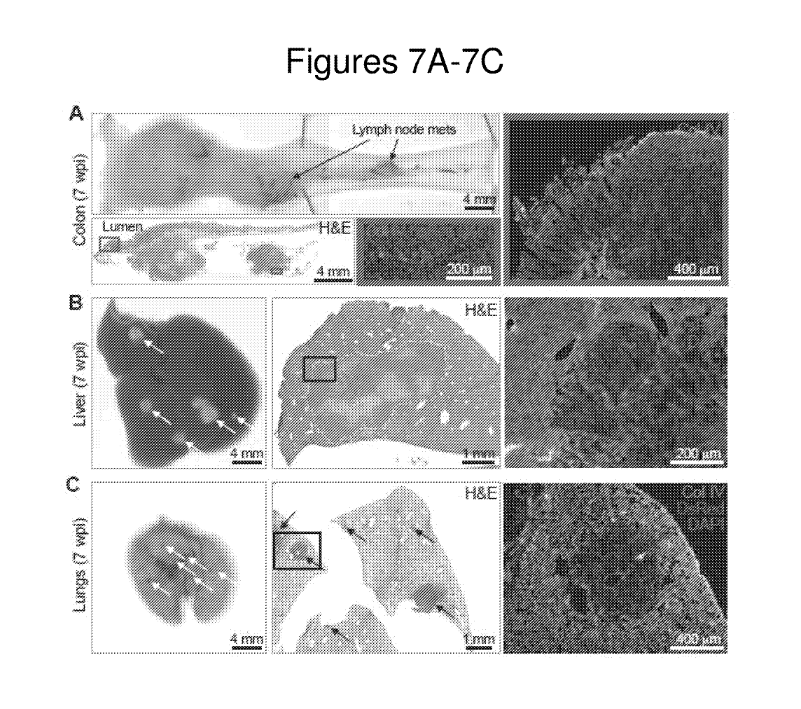

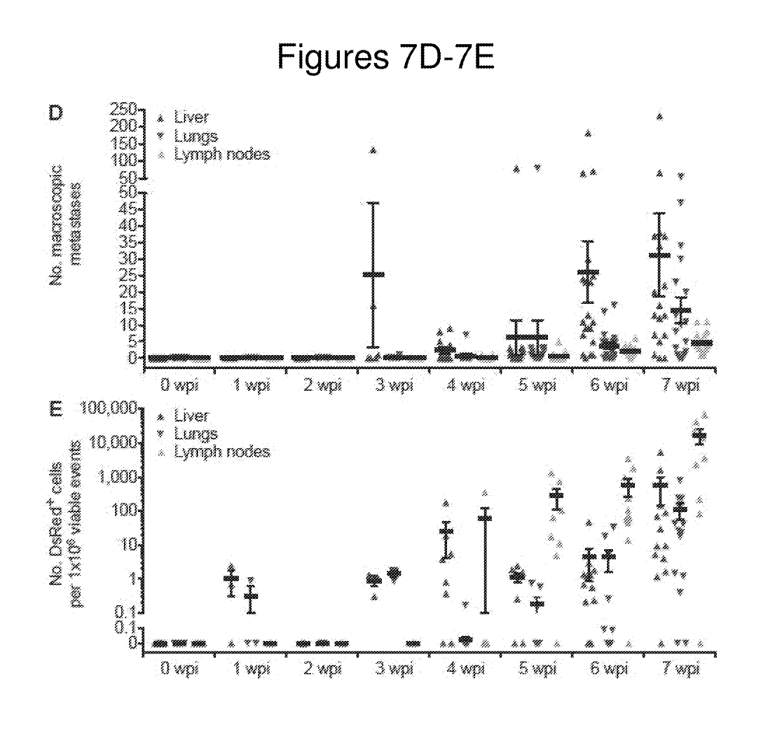

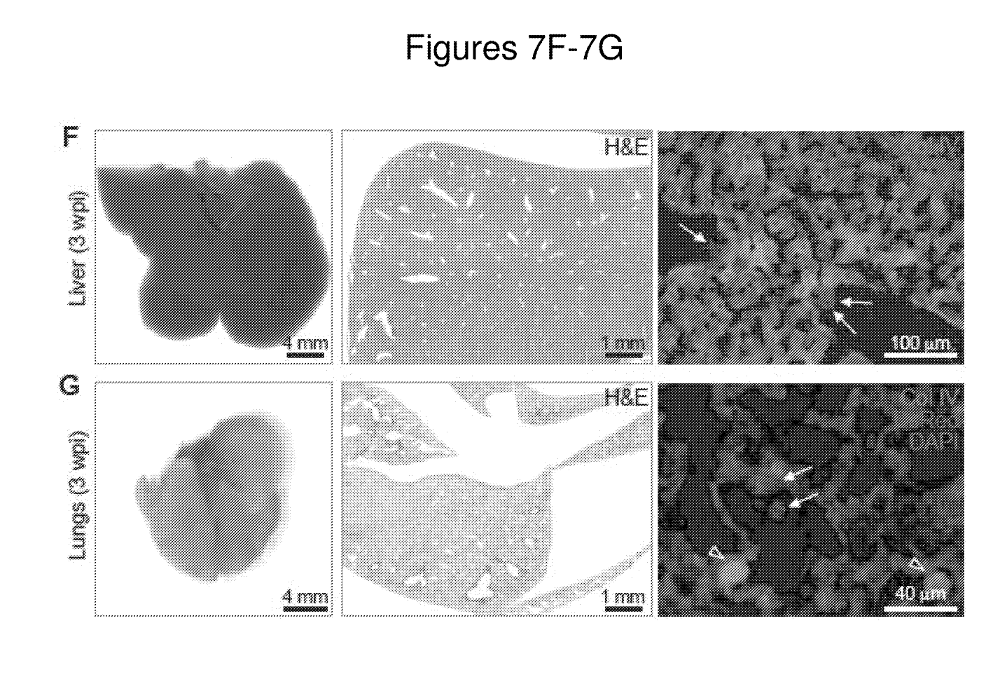

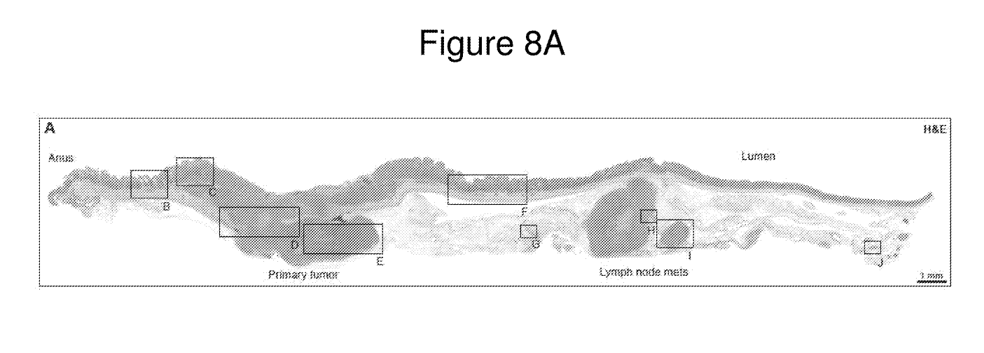

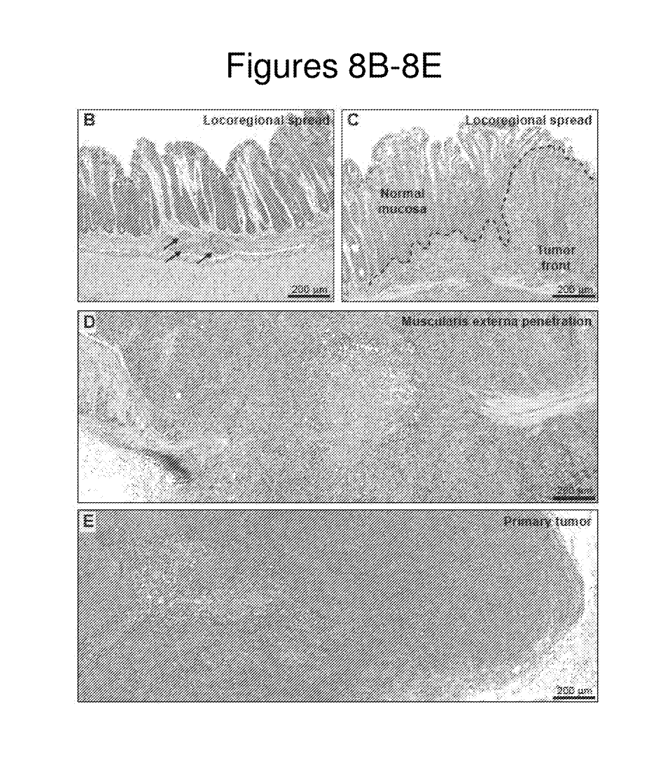

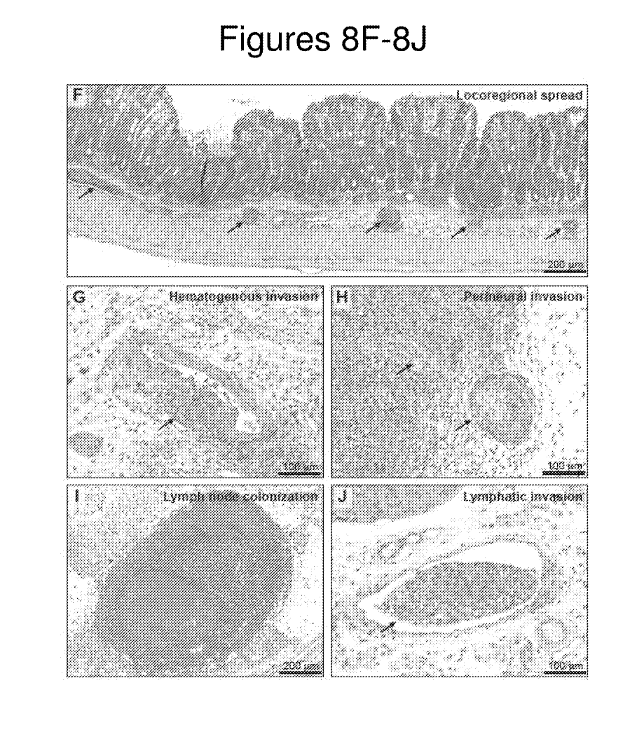

Having developed lumen implantation as a viable technique for generating stage 0 colorectal tumors that progressed to stage II adenocarcinomas, we next assessed tumor-bearing mice for evidence of regional and/or distant metastatic progression corresponding to stage III/IIV disease. At 6-7 wpi, regional intestinal lymph node metastases were detectable as macroscopic tumor nodules located adjacent to the serosa within the draining lymphatic network that ran parallel to the colon wall (FIGS. 7A, 8A, and 8I). Locoregional spread of tumor cells within the colon wall was also prominent, both proximal (FIGS. 8A and 8F) and distal (FIGS. 7A and 8A-8C) to the primary tumor site (FIGS. 8A, 8D, and 8E). Importantly, mice also exhibited hematogenous (FIG. 8G). lymphatic (FIG. 8J) and perineural (FIG. 8H) tumor cell invasion and presented with distant macroscopic, DsRed-positive liver (FIG. 7B) and lung (FIG. 7C) metastases. To better characterize the timing of macroscopic metastasis manifestation, we performed a temporal assessment of metastatic burden via gross examination and determined that macrometastases primarily presented at ˜4 wpi (FIG. 7D). However, macrometastasis presentation does not yield information regarding the precise timing of microscopic disease dissemination. Therefore, we dissociated entire livers, lungs, and intestinal marginal vascular tracts (encompassing the paracolic, intermediate, and principal intestinal lymph nodes) to single cells and monitored for DsRed-positive tumor cells by flow cytometry. Disseminated tumor cells were primarily detectable at ˜3 wpi, one week prior to the manifestation of gross macrometastases (FIG. 7E). We confirmed the presence of disseminated tumor cells within the livers and lungs at 3 wpi by DsRed immunofluorescence, despite the absence of detectable disease at either the gross or histopathological level (FIGS. 7F and 7G). These findings demonstrate lumen implantation of HCT116 colorectal tumors to be a viable in vivo model that metastasizes in a temporal fashion to regional and distant sites relevant to the human disease.

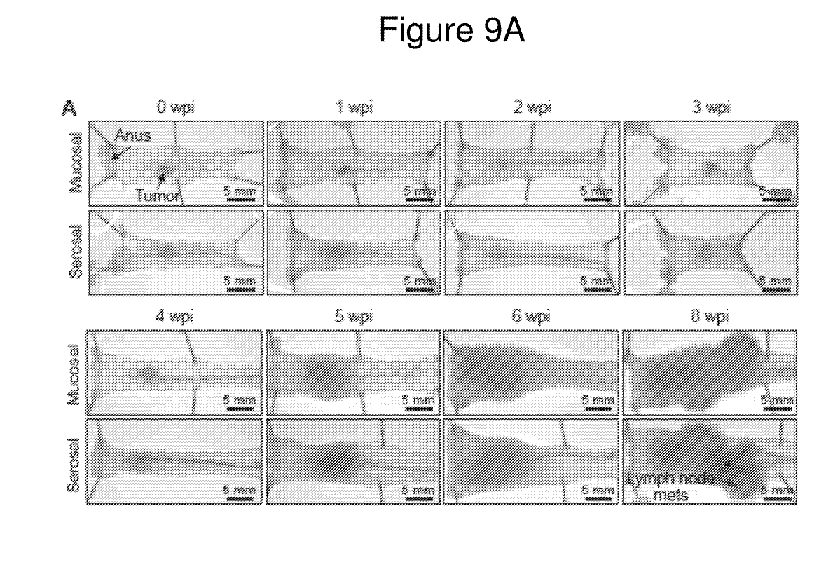

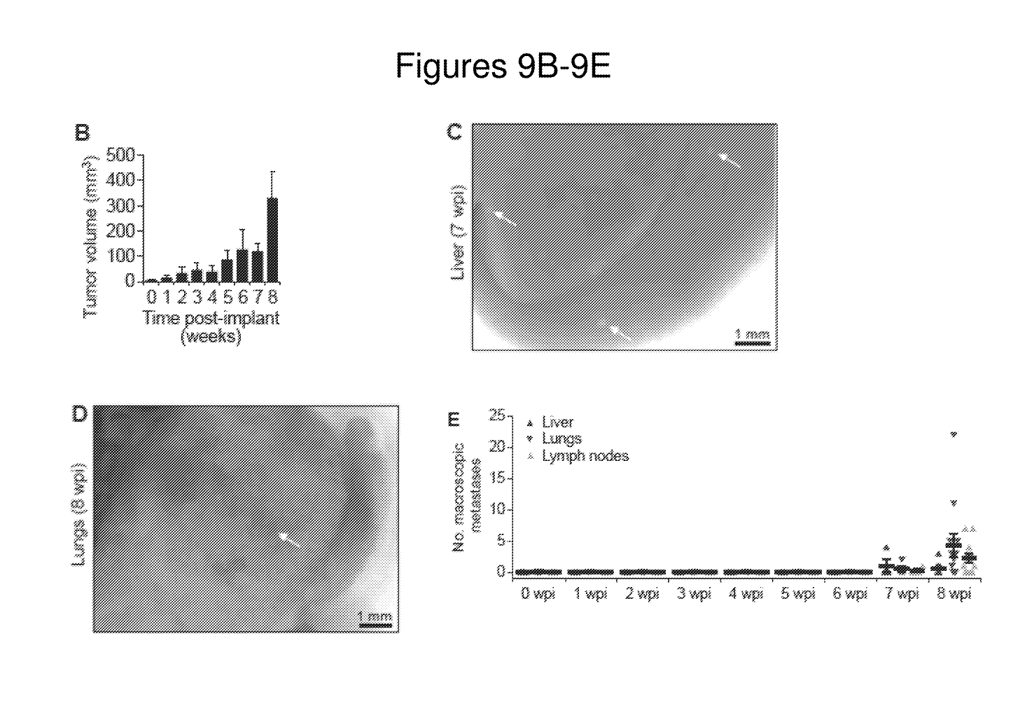

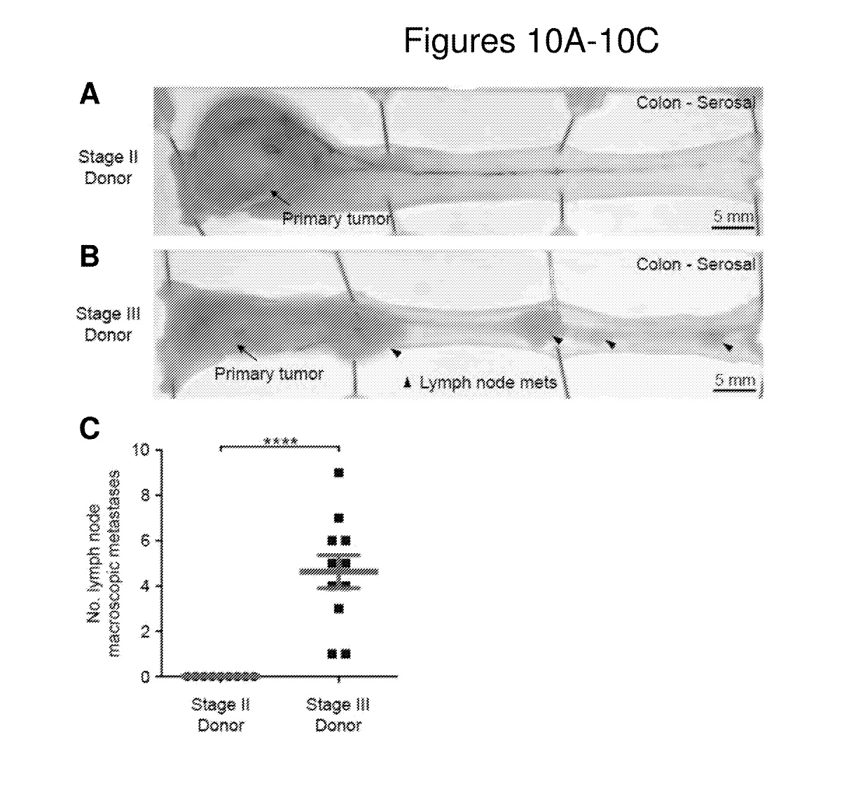

We next determined if the lumen implantation procedure would yield similar progression and metastasis profiles if colorectal tumors of different origin were used as donors. To this end, we used the well-differentiated primary human CRC-derived LS174T cell line. We transduced LS174T cells with a lentivirus encoding DsRed, generated LS174T-DsRed subcutaneous tumors in a donor mouse, and transplanted donor tumor fragments onto the mucosal surface of host mouse colons. Similar to lumen-implanted HCT116 tumors, lumen-implanted LS174T tumors initially grew within the mucosal layer and eventually invaded through the colon wall, such that the bulk of the primary tumor burden was situated on the serosal side of the wall at the 8 wpi harvest endpoint (FIGS. 9A and 9B). Notably, metastatic outgrowths in the liver, lungs, and regional lymph nodes were also apparent in this model, albeit with a longer latency than in the HCT116 tumor-bearing mice (FIGS. 9A and 9C-9E). Using primary human patient CRC tumors as donors, we further demonstrated the utility of this model by showing that lumen-implanted tumors from a stage II patient remained non-metastatic, whereas lumen-implanted tumors from a stage III patient gave rise to intestinal lymph node metastases (FIGS. 10A-10C).

Table 1 summarizes the tumor take rates following lumen implantation of colorectal donor tumors of various types and sources into host mice of various strains using the lumen implantation model (LIM) of CRC. Take rate is defined as the total number of host mice that have undergone successful transplantation of a donor tumor divided by the total number of host mice in which surgical transplantation was attempted, expressed as a percentage. These findings highlight the clinical relevance of disease progression in the LIM.

TABLE 1
Tumor Take Rates Following Lumen Implantation of Colorectal

| | | Host Mouse | Take |
|---|---|---|---|
| Donor Tumor | Donor Source | Strain | Rate |
| Apc^{Min/+}; Kras^{LSLG12D/+}; | Primary polyp | C57BL/6J | 39% |
| Villin-Cre | | | |
| Apc^{Min/+}; Kras^{LSLG12D/+}; | Subcutaneous allograft | C57BL/6J | 60% |
| Villin-Cre | | | |
| HCT116 | Subcutaneous xenograft | NOD/SCID | 44% |
| HCT116 | Subcutaneous xenograft | NSG | 70% |
| LS174T | Subcutaneous xenograft | NOD/SCID | 55% |
| LoVo | Subcutaneous xenograft | NSG | 93% |
| Patient Stage II tumor | Subcutaneous xenograft | NSG | 28% |
| Patient Stage III tumor | Subcutaneous xenograft | NSG | 40% |

### Example 4

### Robust Metastasis to Clinically Relevant Sites Using Lumen Implantation Model of CRC

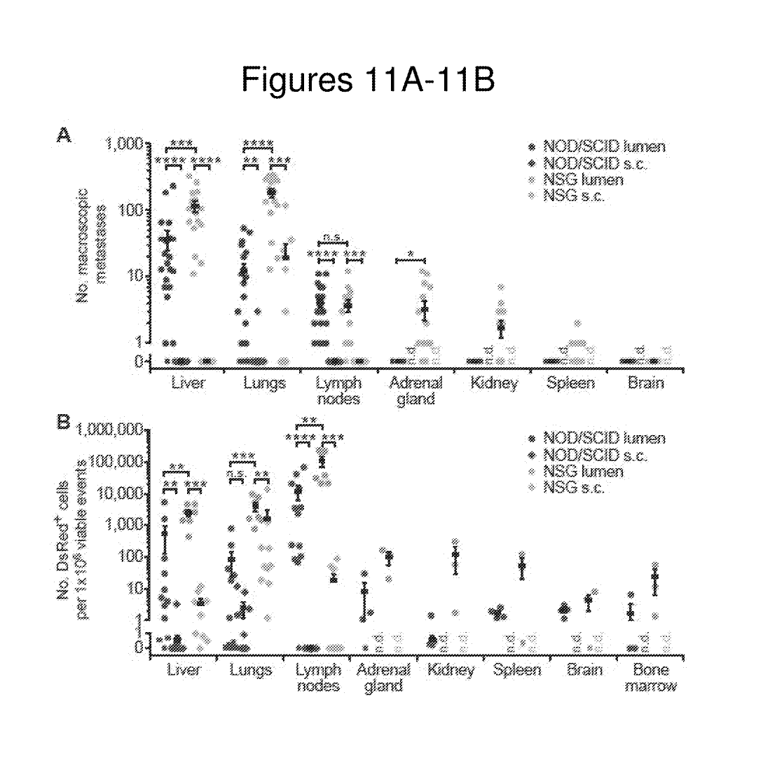

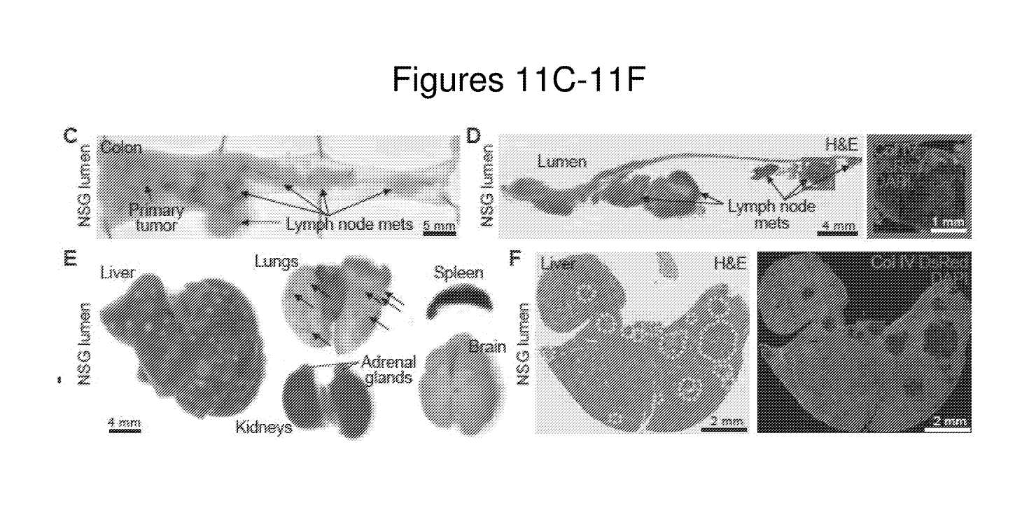

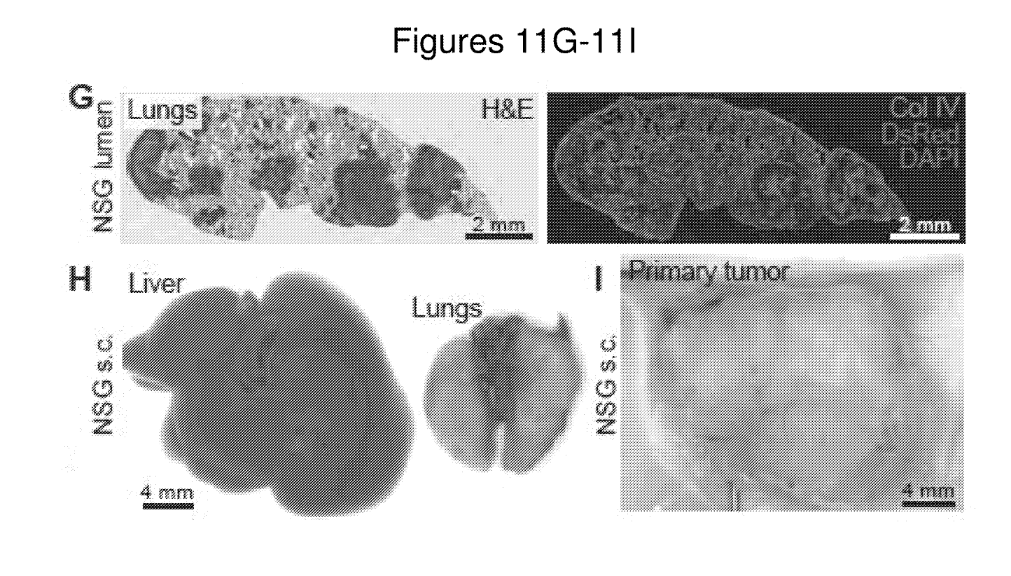

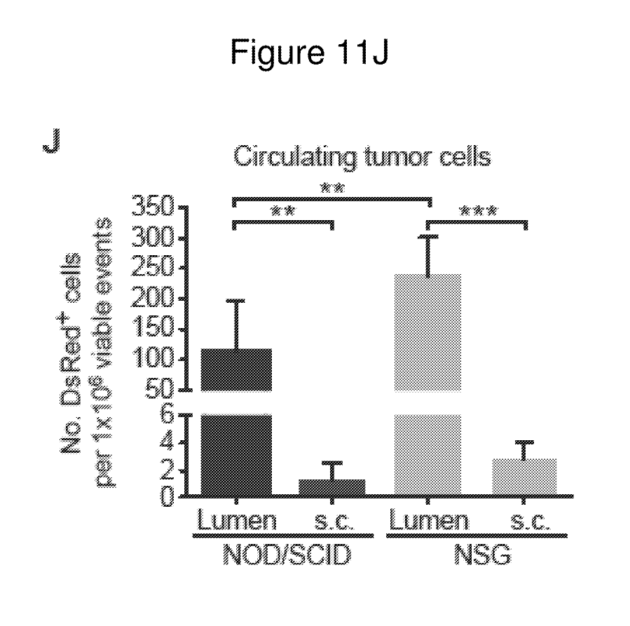

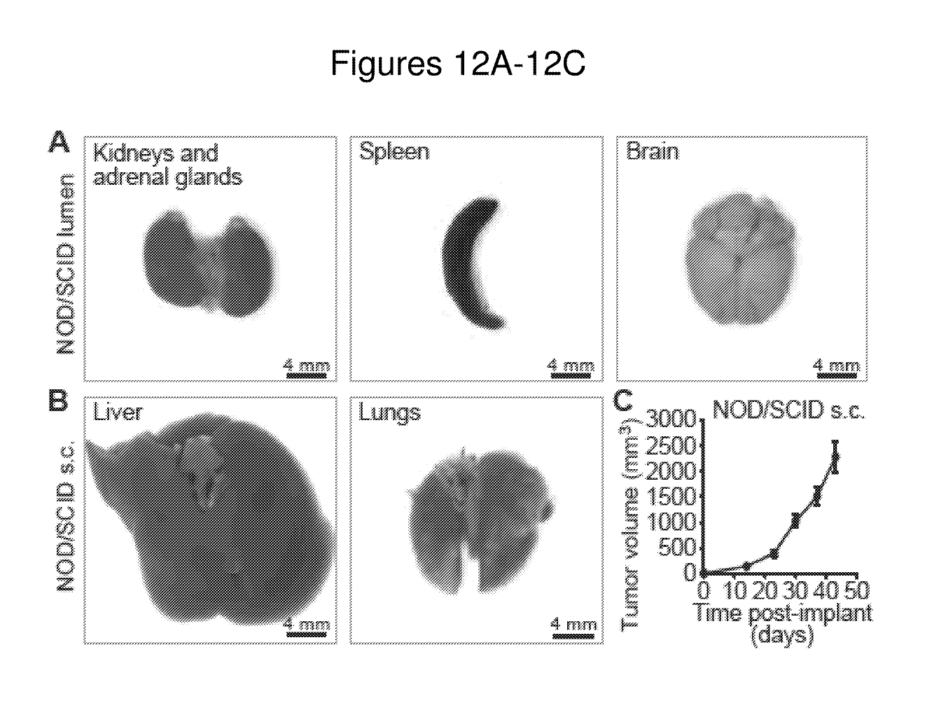

In the clinic, certain tumor types specifically metastasize to certain organs (Fidler et al. Nat. Rev. Cancer. 3 (6): 453-458, 2003). Indeed, CRC predominantly metastasizes to the regional intestinal lymph nodes, liver, and lungs, whereas other organs are largely spared (Chambers et al. Nat. Rev. Cancer. 2 (8): 563-572, 2002). To determine whether the LIM of CRC would accurately recapitulate the preferential target organ specificity observed in humans, we assessed metastatic tumor burden at 6-7 wpi in various internal organs by both macroscopic examination and DsRed-positive tumor cell flow cytometry. NOD/SCID mice bearing lumen-implanted HCT116-DsRed tumors preferentially developed metastases in the liver, lungs, and intestinal lymph nodes, with minimal metastatic burden detectable in the adrenal gland, kidney, spleen, brain, and bone marrow (FIGS. 11A, 11B, and 12A). This preferential target organ homing was maintained when HCT116 tumors were implanted into the colons of the more highly immunocompromised NOD/SCID interleukin-2 receptor gamma chain null (NSG) mouse strain (FIGS. 11C-11G), despite a significant increase in metastatic tumor burden within the liver, lungs, and intestinal lymph nodes in NSG (FIGS. 11A and 11B) compared to NOD/SCID (FIGS. 7B, 7C, 11A, and 11B) mice. This enhancement of primary tumor metastatic potential can be attributed in part to the lack of natural-killer cell activity in NSG mice, consistent with previous reports (Ikoma et al. Oncol. Rep. 14 (3): 633-637, 2005; Quintana et al. Nature. 456: 593-598, 2008). Importantly, we never observed peritoneal carcinomatosis in any of our transplanted NOD/SCID or NSG host mice, a feature that is widespread in the previously reported CRC models to date (Bhullar et al. J. Am. Call. Surg. 213 (1): 54-60; discussion 60-61, 2011. Epub 2011 Mar. 31; Cespedes et al. Am. J. Pathol. 170 (3): 1077-1085, 2007; Fu et at Natl. Acad. Sci. USA. 88 (20): 9345-9349, 1991; Jin et al. Tumour. Biol. 32 (2): 391-397, 2011. Epub 2010 Nov. 19). Given that peritoneal carcinomatosis is not a common manifestation in human CRC (Klaver et al. World. J. Gastroenterl. 18 (39): 5489-5494, 2012), our findings further highlight the relevance and advantage of our metastatic CRC model over those that have been reported in the literature.

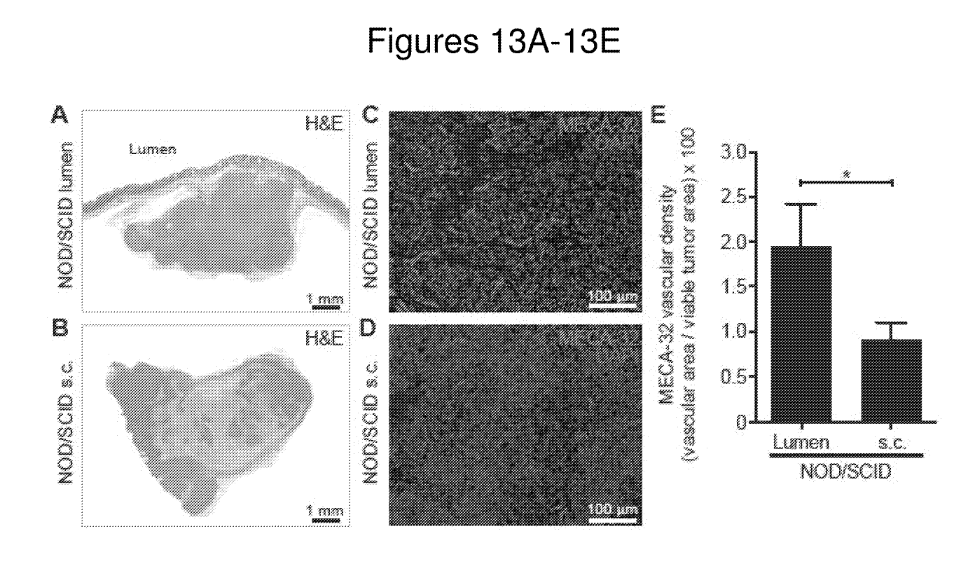

To determine if HCT116 cells were capable of metastasis regardless of implantation site, we implanted HCT116-DsRed cells subcutaneously in both NOD/SCID and NSG mice and assessed metastatic burden. Subcutaneously-implanted tumors did not readily metastasize compared to their lumen-implanted counterparts, in both NOD/SCID (FIGS. 11A, 11B, 12B, and 12C) and NSG (FIGS. 11A, 11B, 11H, and 11I) mouse strains. Similar findings were observed with primary patient colorectal tumor specimens implanted into the subcutaneous and lumen sites. Histological assessment of size-matched and time-matched 6 wpi HCT116 tumors in NOD/SCID mice revealed that whereas subcutaneously-implanted tumors were highly necrotic, lumen-implanted tumors were almost completely devoid of necrosis (FIGS. 13A and 13B). This lack of necrosis may be attributed to enhanced vascularization in lumen implanted tumors, as evidenced by MECA-32 immunostaining for endothelial cells (FIGS. 13C-13E), which in turn may be attributed to enhanced vascular density at the mucosal versus subcutaneous implantation sites. Given that lumen-implanted tumors exhibited increased vascular density concomitant with increased metastasis relative to subcutaneously-implanted tumors, we sought to determine whether circulating tumor cell (CTC) number could be reflective of metastatic potential, as has been reported in the clinic (Cohen et al. J. Clin. Oncol. 26 (19): 3213-3221, 2008). Consistently, lumen-implanted tumors gave rise ˜100-times more CTCs than subcutaneously-implanted tumors in both NOD/SCID and NSG mice, with lumen-implanted NSG mice exhibiting an approximately 2.5-fold greater CTC number than lumen-implanted NOD/SCID mice (FIG. 11J). Hence robust metastasis formation following lumen implantation correlated with increased primary tumor vascularization, which in turn correlated with enhanced tumor cell entry into the circulation. Taken together, these findings do not support an inherent metastatic phenotype in HCT 116 tumors, but rather highlight the fact that the same tumor cells can exhibit drastically different metastatic capacity dependent upon their primary tumor microenvironment.

### Example 5

### Colorectal Cancer Cell Metastasis to the Liver Can Occur Independently of a Lymph Node Metastatic Intermediary

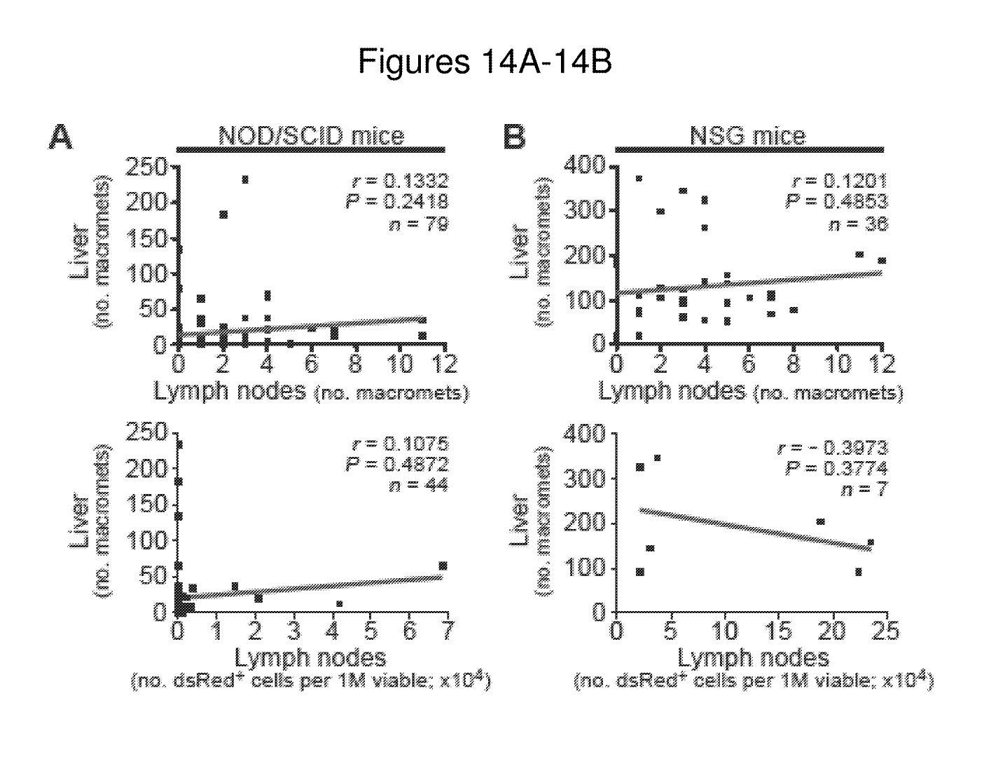

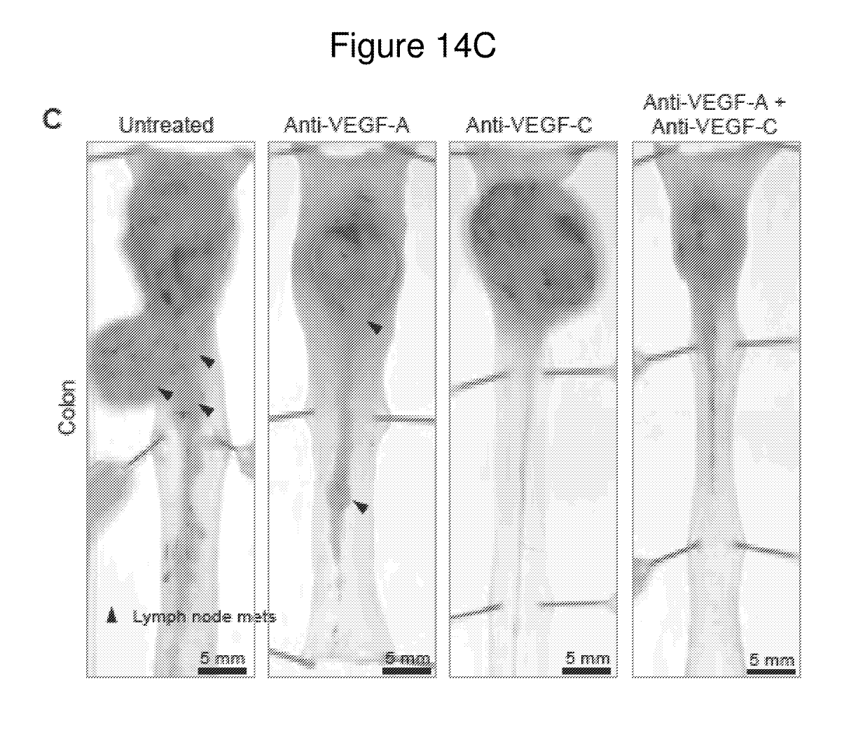

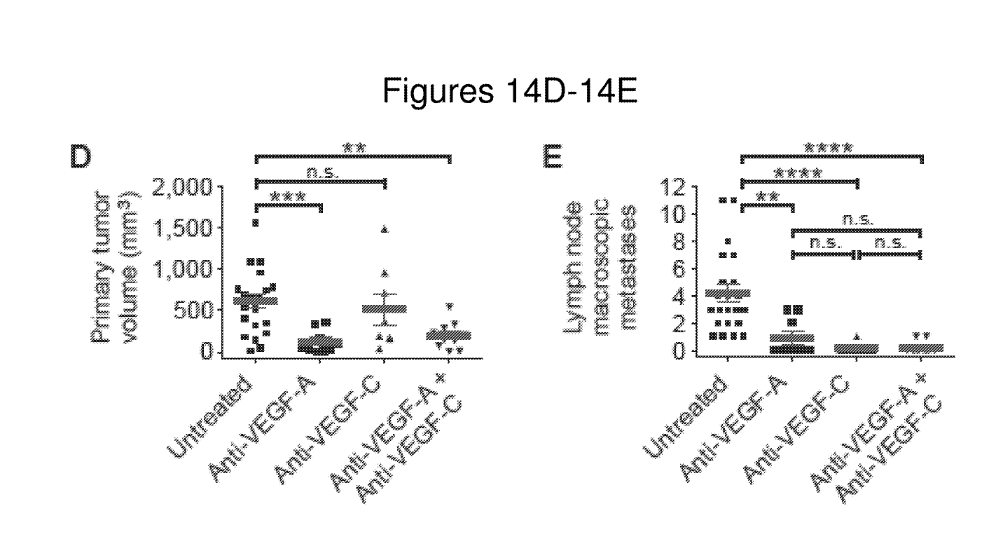

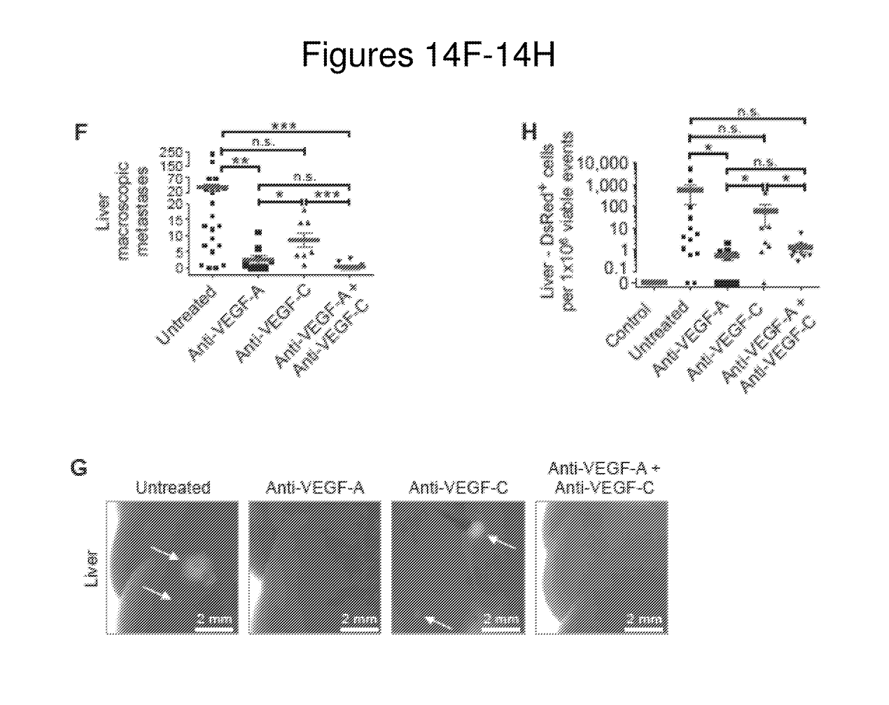

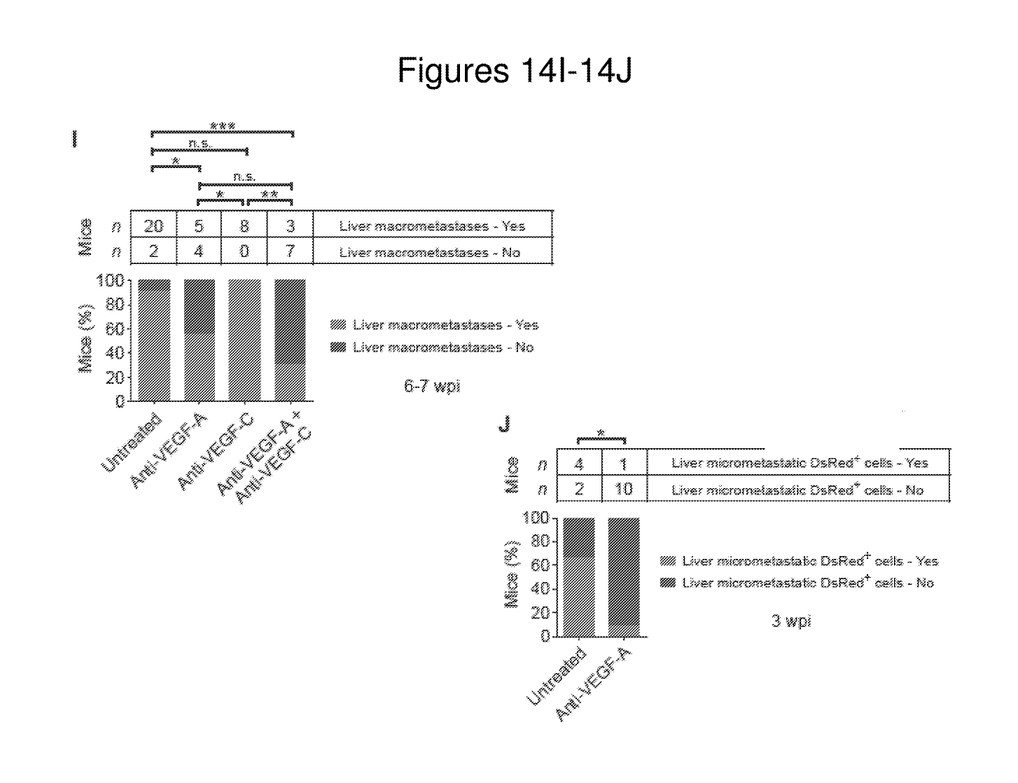

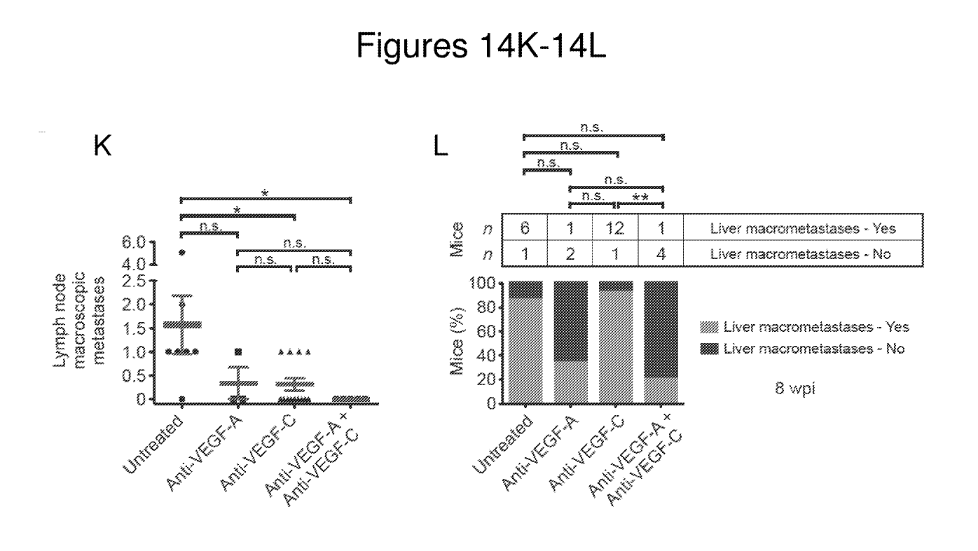

To date, no in vivo model of CRC has demonstrated predictable and reproducible distant metastatic outgrowth within relevant target organs from an orthotopically-established primary colorectal tumor (Heijstek et al. Dig. Surg. 22: 16-25, 2005. Epub 2005 Apr. 14; Kobaek-Larsen et al. Comp. Med. 50 (1): 16-26, 2000; Rosenberg et al. Carcinogenesis. 30 (2): 183-196, 2009. Epub 2008 Nov. 26; Taketo et al. Gastroenterology. 136 (3): 780-798, 2009). The lack of available models has precluded investigations into the route(s) of metastatic spread to distant organs. In the clinic, it is unknown whether colorectal metastases in the liver arise secondary to an initial colonization of the regional intestinal lymph nodes, or whether these liver metastases arise via direct hematogenous spread from the primary tumor, independent of lymph node metastatic growth (Bacac at al. Annu. Rev. Pathol. 3: 221-247, 2008). The first hypothesis is supported by the observations that (i) staging criteria are based on the degree to which the cancer has spread; clinical presentation of intestinal lymph node metastases alone is indicative of stage III disease while the presence of liver metastases are cause for the more advanced stage IV diagnosis (Schwartz et al. Am. J. Health. Syst. Pharm. 65 (11): S8-14, S22-24, 2008), (ii) high co-incidence has been reported for distant liver metastases and intestinal lymph node metastases (Derwinger et al. World. J. Surg. Oncol. 6: 127, 2008), (iii) primary tumor lymphatic vessel density correlates with metastasis to both the lymph nodes and liver (Saad et al. Mod. Pathol. 19 (10): 1317-1323. Epub 2006 Jun. 23), and (iv) tumor cell entry into the lymphatics is presumably easier than entry into the hematogenous vasculature due to a discontinuous basement membrane and a lack of pericyte coverage (Saharinen et al. Trends. Immunol. 25 (7): 387-395, 2004). Several observations support the second hypothesis, including (i) that some CRC patients present with liver metastases in the absence of lymph node involvement (Derwinger et al. World. J. Surg. Oncol. 6: 127, 2008), (ii) that surgical removal of an increased number of draining intestinal lymph nodes in stage III CRC patients may not improve overall survival (Prandi et al. Ann. Surg. 235 (4): 458-463, 2002; Tsikitis et al. J. Am. Coll. Surg. 208 (1): 42-47, 2009; Wong et al. JAMA. 298 (18): 2149-2154, 2007), and (iii) that venous invasion of the primary tumor is an independent prognostic indicator of distant liver metastasis development in CRC (Suzuki et al. Am. J. Surg. Pathol. 33 (11): 1601-1607, 2009). Having developed a LIM as a clinically-relevant model that recapitulates the metastatic tropism of human CRC, we were uniquely positioned to interrogate dissemination routes. In patients a correlation exists between the presence/absence of lymph node metastases and the presence/absence of liver metastases (Derwinger et al. World. J. Surg. Oncol. 6: 127, 2008). In both our NOD/SCID and NSG lumen implantation models, lymph node metastatic burden (both total number of involved lymph nodes and total DsRed-positive tumor cell burden within the lymph nodes) did not correlate with liver metastatic burden (FIGS. 14A and 14B), suggesting that dissemination to the lymph nodes and liver might occur via distinct routes.

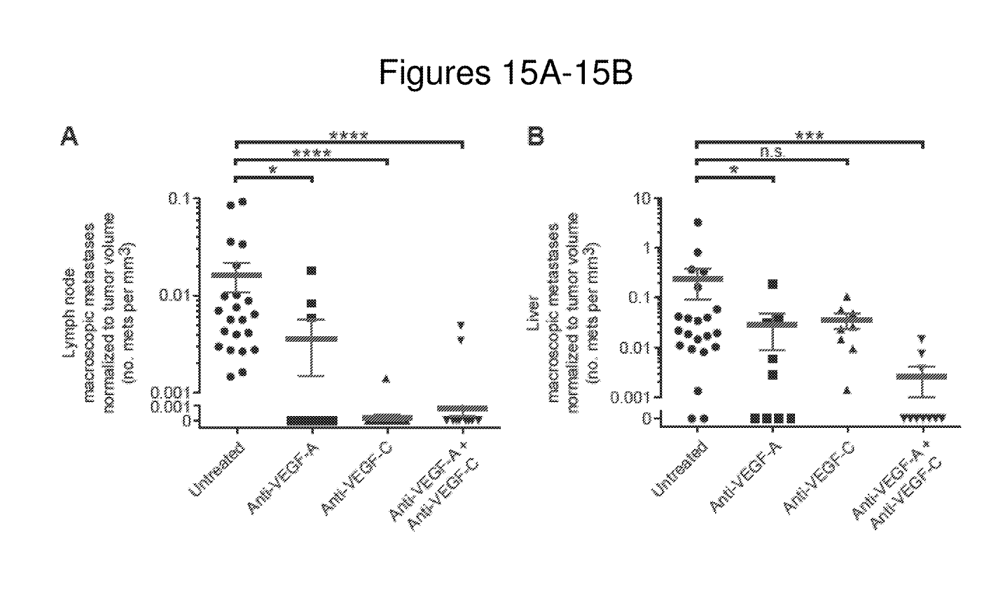

Given that vascular endothelial growth factors (VEGFs) play critical roles in primary tumor vascularization (Carmeliet et al. Nature. 473 (7347): 298-307, 201 1), with VEGF-A and VEGF-C primarily functioning to promote hematogenous and lymphatic vascularization, respectively (Adams et al. Nat. Rev. Mol. Cell. Biol. 8: 464-478, 2007; Oh et al. Dev. Biol. 188: 96-109, 1997), we assessed the effects of function-blocking antibodies against these factors on metastatic dissemination in our model. To this end, we utilized a neutralizing anti-VEGF-A antibody (Liang et al. J. Biol. Chem. 281 (2): 951-961, 2006. Epub 2005 Nov. 7) and generated an anti-VEGF-C antibody. NOD/SCID host mice were treated with antibodies once per week beginning one day prior to HCT116-DsRed or LS174T-DsRed tumor implantation, and metastasis formation was assessed at 6-7 wpi or 8 wpi, respectively. Anti-VEGF-A inhibited macroscopic metastasis formation in the liver (FIGS. 14F and 14G), which was confirmed by reductions in both DsRed-positive tumor cell burden (FIG. 14H) and the percentage of mice that presented with liver involvement (FIG. 14I). Anti-VEGF-A also attenuated (FIGS. 14E and 14K) but did not eliminate (FIG. 14C) the growth of gross lymph node metastases, consistent with its role in supporting the growth of metastatic lymph node tumors by promoting angiogenesis (Niki et al. Clin. Cancer. Res. 6: 2431-2439, 2000). In contrast, despite a near complete abrogation of lymph node metastases (FIGS. 14E and 14K), anti-VEGF-C did not significantly inhibit liver metastasis formation (FIGS. 14F and 14G) and accordingly did not reduce DsRed-positive tumor cell burden within the liver (FIG. 14H). Anti-VEGF-C also had no effect on decreasing the percentage of mice that presented with liver macrometastases in either HCT116 or LS174T LIM (FIGS. 14I and 14L). Combination treatment with anti-VEGF-A and anti-VEGF-C antibodies inhibited both lymph node and liver metastasis formation (FIGS. 14E-14I and 14L). To account for differences in primary tumor volume following antibody treatment (FIGS. 14C and 14D), we normalized liver metastatic burden to primary tumor volume in all treatment arms and confirmed that anti-VEGF-C-mediated blockade of lymph node metastasis had no impact on liver metastasis formation (FIGS. 15A and 15B). Although our data support a role for VEGF-A but not VEGF-C in CRC liver metastasis formation, whether anti-VEGF-A inhibited the outgrowth of tumor cells that had already seeded the liver or whether anti-VEGF-A directly inhibited primary tumor cell dissemination to the liver remained uncertain. To answer this question, we treated tumor-bearing mice with anti-VEGF-A and assessed livers at 3 wpi, prior to the manifestation of macroscopic liver metastases (FIGS. 7D and 7E), for micrometastatic DsRed-positive tumor cells by flow cytometry. Anti-VEGF-A significantly reduced the number of mice with detectable disseminated tumor cells within the liver (FIG. 14J). Taken together, these findings demonstrate that CRC cell metastasis from the primary tumor to the liver can occur via direct hematogenous spread, independent of a lymph node metastatic intermediary.

### Other Embodiments

While the invention has been described in connection with specific embodiments thereof, it will be understood that it is capable of further modifications and this application is intended to cover any variations, uses, or adaptations of the invention following, in general, the principles of the invention and including such departures from the present disclosure that come within known or customary practice within the art to which the invention pertains and may be applied to the essential features hereinbefore set forth.

All patents, patent applications, patent application publications, and other publications cited or referred to in this specification are herein incorporated by reference to the same extent as if each independent patent, patent application, patent application publication or publication was specifically and individually indicated to be incorporated by reference. Such patent applications specifically include U.S. Provisional Patent Application Nos. 61/857,638, filed Jul. 23, 2013, and 61/954,788, filed Mar. 18, 2014, from which this application claims benefit.

1. A rodent comprising a donor tumorigenic cell implant on its colonic mucosal surface, wherein implantation does not result in breach of the colon wall.

2. The rodent of claim 1, wherein the donor tumorigenic cell implant is capable of invasive growth through the colon wall to the colonic serosal surface.

3. The rodent of claim 2, wherein the invasive growth of the donor tumorigenic cell implant is characterized by metastases in the intestinal lymph nodes, liver, or lungs.

4. The rodent of claim 1, wherein the rodent does not exhibit detectable tumor formation in the peritoneal cavity post-implantation.

5. The rodent of claim 1, wherein the donor tumorigenic cell implant comprises cells of a cancer cell line.

6. The rodent of claim 5, wherein the cancer cell line is a colorectal cancer (CRC) cell line.

7. The rodent of claim 1, wherein the donor tumorigenic cell implant is an intact tumor, or fragment thereof.

8. The rodent of claim 7, wherein the intact tumor, or fragment thereof, is an intact malignant tumor, or fragment thereof.

9. The rodent of claim 7, wherein the intact tumor, or fragment thereof, is an intact benign tumor, or fragment thereof.

10. The rodent of claim 7, wherein the intact tumor, or fragment thereof, is an intact colorectal tumor, or fragment thereof.

11. The rodent of claim 7, wherein the intact tumor, or fragment thereof, is an intact non-colorectal tumor, or fragment thereof.

12. The rodent of claim 1, wherein the rodent is a mouse.

13. The rodent of claim 12, wherein the mouse is immunodeficient.

14. The rodent of claim 13, wherein the immunodeficient mouse is a nod/scio mouse or a NOD/SCID/interleukin-2 receptor gamma chain null (NSG) mouse.

15. A method for generating a rodent model for colorectal cancer, the method comprising:
(a) exteriorizing the colonic mucosal surface of a host rodent;
(b) implanting one or more tumorigenic cells onto the colonic mucosal surface; and
(c) re-inserting the exteriorized colon comprising the one or more implanted tumorigenic cells into the host rodent,
thereby generating a rodent model for colorectal cancer.

16. The method of claim 15, wherein the tumorigenic cells are capable of invasive growth through the colon wall to the colonic serosal surface.

17. The method of claim 15, wherein the rodent model for colorectal cancer is characterized by metastases of the one or more implanted tumorigenic cells in the intestinal lymph nodes, liver, or lungs.

18. The method of claim 15, wherein the rodent model does not exhibit detectable tumor formation in the peritoneal cavity post-implantation.

19. The method of claim 15, wherein the one or more tumorigenic cells are one or more donor tumorigenic cells.

20. The method of claim 15, wherein the one or more tumorigenic cells are in an intact tumor, or fragment thereof.

21. The method of claim 20, wherein the one or more tumorigenic cells are from a cancer cell line.

22. The method of claim 15, wherein the rodent is a mouse.

23. A method of screening for a compound that inhibits growth of tumorigenic cells, the method comprising:
(a) contacting the donor tumorigenic cell implant of a rodent of claim 1 with a candidate compound in the rodent; and
(b) determining whether the candidate compound inhibits growth of the tumorigenic cells, thereby identifying the candidate compound as a compound that inhibits growth of tumorigenic cells.

24. A method of screening for an adjuvant that inhibits growth of tumorigenic cells, the method comprising:
(a) removing the donor tumorigenic cell implant from the colonic mucosal surface of the rodent of claim 1;
(b) administering to the rodent a candidate compound; and
(c) determining whether the candidate compound inhibits growth of tumorigenic cells,
Thereby identifying the candidate compound as an adjuvant that inhibits growth of tumorigenic cells.

25. The method of claim 23, wherein determining whether the candidate compound inhibits growth of tumorigenic cells comprises evaluating the ability of the candidate compound to evoke at least one response selected from the group consisting of: reduction or stabilization in the number of tumorigenic cells; reduction or stabilization of tumor size; reduction or stabilization of tumor load; reduction or stabilization of tumorigenic cell invasiveness; and reduction or stabilization of tumor metastasis.

26. The method of claim 23, wherein the candidate compound is a small molecule, a peptide, a polypeptide, an antibody, an antibody fragment, or an immunoconjugate.

27. The method of claim 23, wherein the donor tumorigenic cell implant is capable of invasive growth through the colon wall to the colonic serosal surface.

28. The method of claim 23, wherein the invasive growth of the donor tumorigenic cell implant is characterized by metastases in the intestinal lymph nodes, liver, or lungs.

29. The method of claim 23, wherein the rodent does not exhibit detectable tumor formation in the peritoneal cavity post-implantation.

30. The method of claim 23, wherein the rodent is a mouse.
